(19) United States
(12) Patent Application Publication
(10) Pub. No.: US 2018/0192868 A1
(43) Pub. Date: Jul. 12, 2018

(54) OPHTHALMIC EXAMINATION SYSTEM

(71) Applicant: Topcon Corporation, Tokyo (JP)

(72) Inventors: Tomohiro SAKURADA, Tokyo (JP); Tatsuro YOKOI, Kuki-shi (JP)

(73) Assignee: Topcon Corporation, Tokyo (JP)

(21) Appl. No.: 15/864,077

(22) Filed: Jan. 8, 2018

(30) Foreign Application Priority Data
Jan. 11, 2017 (JP) 2017-002632

(51) Int. Cl. A61B 3/032; A61B 3/00; G06F 3/0484; G06F 3/14

(52) U.S. Cl. CPC A61B 3/032; A61B 3/0033; H04L 12/28; G06F 3/04842; G06F 3/1454; A61B 3/0041

(57) ABSTRACT

An ophthalmic examination system of an exemplary embodiment includes a plurality of ophthalmic examination apparatuses, a plurality of examiner terminals, a communication establishing device, an information transmitting device, and an output device. The communication establishing device is capable of establishing communication between at least three apparatuses including at least one of the plurality of ophthalmic examination apparatuses and at least two of the plurality of examiner terminals. The information transmitting device transmits at least part of information transmitted from one of the at least three apparatuses between which the communication has been established by the communication establishing device, to at least one of other two or more apparatuses. The output device outputs information transmitted by the information transmitting device.

### CROSS-REFERENCE TO RELATED APPLICATION

This application is based upon and claims the benefit of priority from Japanese Patent Application No. 2017-002632, filed Jan. 11, 2017; the entire contents of which are incorporated herein by reference.

### FIELD

Embodiments described herein relate to an ophthalmic examination system including a plurality of ophthalmic examination apparatuses and a plurality of examiner terminals.

### BACKGROUND

Ophthalmic examination apparatuses are used for the examinations (measurements of properties, imaging, etc.) of eyes in medical institutions, optician's stores, health check and screening venues, patient's home, and the like. Typical examples of the ophthalmic examination apparatuses include the followings.
Visual acuity test apparatuses for measuring visual acuity based on responses to visual targets presented
Eye refraction test apparatuses (refractometers, keratometers) for measuring the refractive properties of eyes
Tonometers for measuring the intraocular pressure
Specular microscopes for obtaining the properties of corneas (conical thickness, cell distribution, etc.)
Wavefront analyzers for obtaining the aberration information of eyes using a Hartmann-Shack sensor
Perimeters and micro-perimeters for the detection of visual field defects
Optical coherence tomography (OCT) apparatuses for obtaining cross sectional images, three dimensional data, analysis data, etc. of eye funduses, anterior eye segments, etc. using optical interference
Fundus cameras for photographing eye funduses
Scanning laser ophthalmoscopes (SLO) for capturing images of eye funduses by laser scanning using a confocal optical system
Multifunctional apparatuses having a combination of two or more functions

It is often the case that the examiner instructs a subject and operates the ophthalmic examination apparatus during an eye examination. In recent years, remote examination has been becoming popular. In the remote examination, the examiner, who is not present at the place where the ophthalmic examination apparatus is installed, conducts the examination while providing instructions for the subject and the ophthalmic examination apparatus (for example, see Japanese Unexamined Patent Application Publication No. 2001-286442, Japanese Unexamined Patent Application Publication No. 2002-10978, Japanese Unexamined Patent Application Publication No. 2002-78679, Japanese Unexamined Patent Application Publication No. 2002-78681, Japanese Unexamined Patent Application Publication No. 2005-342042, and International Publication No. 2003/041572). The instructions for the subject include visual instructions and auditory instructions.

### BRIEF DESCRIPTION OF THE DRAWINGS

FIG. 1A is a schematic diagram illustrating an example of the configuration of an ophthalmic examination system according to an exemplary embodiment.

FIG. 1B is a schematic diagram illustrating an example of the configuration of the ophthalmic examination system according to the exemplary embodiment.

FIG. 2 is a schematic diagram illustrating an example of the configuration of the ophthalmic examination system according to the exemplary embodiment.

FIG. 3 is a schematic diagram illustrating an example of the configuration of the ophthalmic examination system according to the exemplary embodiment.

FIG. 4 is a schematic diagram illustrating an example of the configuration of the ophthalmic examination system according to the exemplary embodiment.

FIG. 5 is a schematic diagram illustrating an example of the configuration of the ophthalmic examination system according to the exemplary embodiment.

FIG. 6 is a sequence diagram illustrating an example of the usage mode of the ophthalmic examination system according to the exemplary embodiment.

FIG. 7A is a schematic diagram for describing the usage mode of the ophthalmic examination system according to the exemplary embodiment.

FIG. 7B is a schematic diagram for describing the usage mode of the ophthalmic examination system according to the exemplary embodiment.

FIG. 7C is a schematic diagram for describing the usage mode of the ophthalmic examination system according to the exemplary embodiment.

FIG. 8 is a sequence diagram illustrating an example of the usage mode of the ophthalmic examination system according to the exemplary embodiment.

FIG. 9 is a schematic diagram illustrating a modification example of the ophthalmic examination system according to the exemplary embodiment.

FIG. 10 is a schematic diagram illustrating an example of the display screen provided by the ophthalmic examination system according to the exemplary embodiment.

### DETAILED DESCRIPTION

In the remote examination, instructions are sent to a plurality of ophthalmic examination apparatuses distributed in various places. In order to efficiently implement a remote examination thus performed, it is necessary to construct a system assuming various situations.

For example, it is desirable to issue an appropriate instruction at an appropriate timing according to the degree of understanding of the examination contents by the subject. In the case where an examiner accompanies the subject as in the conventional cases, the examiner can assist the subject by being there to perceive the state of the subject and the progress state of the examination on the spot. However, it is very difficult to put such an assistance into practice with conventional remote examination techniques.

In addition, the examiner's degree of expertise and level of skill largely influence the examination to proceed accurately and smoothly; however, it is very difficult in practice to assign such a high-skilled examiner full-time or to every examination facility.

According to some embodiments, it is possible to deal with various situations occurring in remote examinations.

In one embodiment, an ophthalmic examination system includes the followings: a plurality of ophthalmic examination apparatuses; a plurality of examiner terminals; a communication establishing device capable of establishing communication between at least three apparatuses including at least one of the plurality of ophthalmic examination apparatuses and at least two of the plurality of examiner terminals; an information transmitting device that transmits at least part of information transmitted from any of the at least three apparatuses between which the communication has been established by the communication establishing device, to at least one of other two or more apparatuses; and an output device that outputs information transmitted by the information transmitting device.

Referring now to the drawings, exemplary embodiments are described below. All publications referred to herein and any known technology and technique are hereby incorporated by reference in their entireties.

According to one embodiment, an ophthalmic examination system is applied to remote examination by means of ophthalmic examination apparatuses installed in various facilities, portable ophthalmic examination apparatuses, or the like. In the remote examination according to the embodiment, various examiners assist the examination. There are internal examiners and external examiners as types of examiners.

An internal examiner assists the examination inside the facility where the ophthalmic examination apparatus is installed. Typically, the internal examiner perceives the state of all or some subjects undergoing examinations in the facility and sends instructions using a tablet terminal or a wearable device (for example, a wireless earphone). The subjects can proceed with the examination based on instructions from the internal examiner. An information processing apparatus used by the internal examiner is called an internal examiner terminal.

An external examiner assists the examination from the outside of the facility where the ophthalmic examination apparatus is installed. Typically, the system can be configured in such a manner that the external examiner performs assistance from a facility for managing the examinations (i.e., from a management center) or from the outside of the management center. Examples of locations outside the management center include the examiner's home. The external examiner perceives the state of all or some subjects currently undergoing examinations and sends instructions by using an information processing apparatus installed in the management center or the examiner's home. The subject can proceed with the examination based on the instructions sent from the external examiner. The information processing apparatus used by the external examiner is called an external examiner terminal.

Examples of the facilities where the ophthalmic examination apparatuses are installed include medical institutions, optician's stores, health check and screening venues, patient's home, welfare facilities, public facilities, examination cars, and the like. In other words, the facilities where the ophthalmic examination apparatuses are installed may include one or both of the fixed facilities such as medical institutions and mobile facilities such as examination cars. Further, the instructions sent from the examiner to the subject may include assistance (or support) to the subject about how to proceed with the examination, how to use the ophthalmic examination apparatus, advices to the subject, and the like. In addition, an examiner can send instructions to other examiners. Typically, an external examiner who is an expert can assist an internal examiner who is an unskilled person.

The ophthalmic examination apparatus may be any kind of apparatus utilized for eye examination and may have one or both of the function of an ophthalmic measurement apparatus and the function of an ophthalmic imaging apparatus. The ophthalmic measurement apparatus is an apparatus for measuring the properties of the subject's eye. Examples of the ophthalmic measurement apparatus include visual acuity test apparatuses (visual target presenting apparatuses, phoropters, etc.), eye refraction test apparatuses (refractometers, keratometers, etc.), tonometers, specular microscopes, wave front analyzers, perimeters, micro perimeters, and the like. The ophthalmic imaging apparatus is an apparatus for imaging the subject's eye. Examples of the ophthalmic imaging apparatus include OCT apparatuses, fundus cameras, SLOs, and the like. The ophthalmic examination apparatus may be provided with application software for processing and/or analyzing measurement data, captured images, or the like.

### <Configuration of the Ophthalmic Examination System>

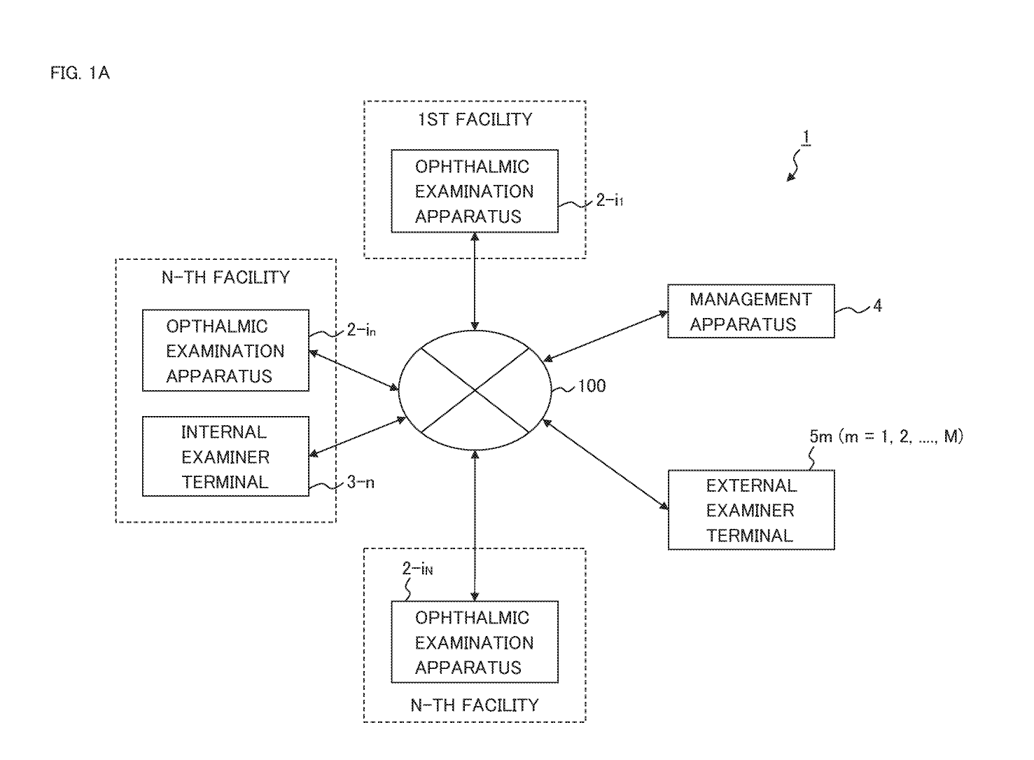

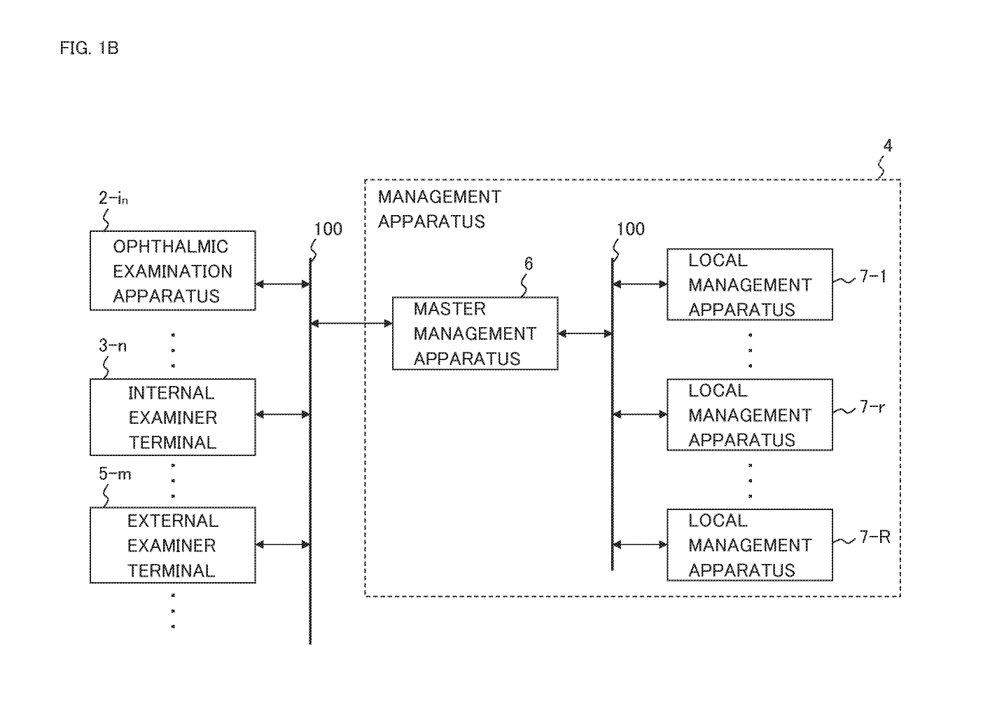

Described below is an example of the configuration of the ophthalmic examination system according to an exemplary embodiment. The ophthalmic examination system 1 illustrated in FIG. 1A as an example is configured by using a network (the communication line 100) that connects N facilities (first to N-th facilities) in which examination is performed, the management apparatus 4, and the external examiner terminal(s) 5m (where m=1 to M, M is an integer 1 or larger).

Each of the facilities (n-th facility: where n=1 to N, N is an integer 1 or larger) is provided with one or more ophthalmic examination apparatus(es) 2-iₙ (where iₙ=1 to Kₙ, Kₙ is an integer 1 or larger). The ophthalmic examination apparatus 2-iₙ is included in the ophthalmic examination system 1. Incidentally, the ophthalmic examination system 1 may include an examination apparatus capable of examination other than ophthalmic examination.

The ophthalmic examination apparatus 2-iₙ of the present example have the function of an “examination apparatus” that performs the examination of the subject (examination of the subject's eye), and the function of a “computer” that performs various types of data processing and communicates with external devices. For another example, the examination apparatus and the computer may be provided separately. In this case, the examination apparatus and the computer are capable of communicating with each other. There may be arbitrary number of examination apparatuses and arbitrary number of computers. For example, there may be a single computer and a plurality of examination apparatuses.

At least one of the N facilities (the first to N-th facilities) is provided with an information processing apparatus used by an internal examiner (i.e., internal examiner terminal). In the example illustrated in FIG. 1A, no internal examiner terminal is installed in the first facility and the N-th facilities, and the internal examiner terminal 3-n is installed in the n-th facility.

The internal examiner terminal 3-n is a computer for use in the n-th facility. The internal examiner terminal 3-n may include at least one of a mobile terminal such as a tablet terminal of a smartphone, a wearable device such as a wireless earphone, and a server installed in the n-th facility (e.g., in-house server). The internal examiner terminal 3-n is only required to be a computer capable of realizing its function in the n-th facility. The internal examiner terminal 3-n may include, for example, a computer installed outside the n-th facility (e.g., cloud server).

The ophthalmic examination apparatus 2-iₙ and the internal examiner terminal 3-n may be capable of communicating with each other through a network such as a network built in the n-th facility (e.g., in-house LAN), a wide area network (e.g., the Internet), or near-field communication technology.

The ophthalmic examination apparatus 2-iₙ may include the function as a server. In the case where this configuration is applied, the ophthalmic examination apparatus 2-iₙ and the internal examiner terminal 3-n may be configured to communicate directly with each other. In this case, there is no need to provide the function to perform communication between the internal examiner terminal 3-n and a management apparatus 4. In other words, the system may be configured in such a manner that the communication between the management apparatus 4 and the internal examiner terminal 3-n is performed via the ophthalmic examination apparatus 2-iₙ.

The ophthalmic examination system 1 may include the management apparatus 4. Typically, the management apparatus 4 is installed in the management center. The management apparatus 4 can communicate with the external examiner terminal(s) 5m via the communication line 100 (e.g., LAN, wide area network). Further, the management apparatus 4 can communicate with the ophthalmic examination apparatuses 2-iₙ installed in the first to the N-th facilities via the communication line 100 (e.g., wide area network).

The management apparatus 4 controls communication between at least three apparatuses including at least one of the plurality of ophthalmic examination apparatuses 2-iₙ and at least two of the plurality of examiner terminals (including the internal examiner terminals 3-n and the external examiner terminals 5m). For example, the management apparatus 4 controls communication between three apparatuses including one of the plurality of ophthalmic examination apparatuses 2-iₙ, the internal examiner terminal 3-n, and the external examiner terminal 5m. In another example, the management apparatus 4 controls communication between three apparatuses including one of the plurality of ophthalmic examination apparatuses 2-iₙ, and two of the internal examiner terminals 3-n. In yet another example, the management apparatus 4 controls communication between three apparatuses including one of the plurality of ophthalmic examination apparatuses 2-iₙ, and two of the external examiner terminals 5m. It should be noted that the number of apparatuses on which communication control is performed by the management apparatus 4 is not limited to three. Therefore, the management apparatus 4 can control communication between four or more apparatuses.

The management apparatus 4 may have at least one of the functions of, for example, relaying communication between the ophthalmic examination apparatus 2-iₙ and the examiner terminal, relaying communication between two examiner terminals, and relaying communication between two ophthalmic examination apparatuses 2-iₙ. Further, the management apparatus 4 may have the function of recording the contents of the communication. In addition, the management apparatus 4 may be provided with the function of associating the ophthalmic examination apparatus 2-iₙ and the examiner terminal with each other, that is, the function of assigning an examiner to each of the ophthalmic examination apparatuses 2-iₙ. The correspondence between the ophthalmic examination apparatuses 2-iₙ and examiner terminals may be any of the one-to-one correspondence, many-to-one correspondence, and one-to-many correspondence. The management apparatus 4 may be configured to have the function of detecting the occurrence of a predetermined error in the contents of the communication between the ophthalmic examination apparatus 2-iₙ and the examiner terminal, the function of recording the error occurrence, and the function of notifying the examiner terminal and/or other examiner terminal(s) of a warning corresponding to the error occurrence.

The management apparatus 4 includes one or more computers. FIG. 1B illustrates an example of a case in which the management apparatus 4 includes a plurality of computers. The management apparatus illustrated in FIG. 1B includes the master management apparatus 6 and the R number of local management apparatuses 7-r (where R is an integer 2 or larger). The master management apparatus 6 and each local management apparatus 7-r can communicate with each other via the communication line 100. Further, two or more local management apparatuses 7-r may communicate with one another via the communication line 100.

The master management apparatus 6 can communicate via the communication line 100 with the apparatuses installed in each of the first to the N-th facilities. The apparatuses installed in each of the first to the N-th facilities include at least the ophthalmic examination apparatuses 2-iₙ, and may further include the internal examiner terminals 3-n. The master management apparatus 6 is installed inside or outside the management center. Further, the master management apparatus 6 may include two or more information processing apparatuses.

In one example, two or more management centers may be provided. The local management apparatus 7-r is installed, for example, in one of the management centers. In the case where one or more external examiner terminals 5m are installed in the management center, the local management apparatus 7-r can manage each of the one or more external examiner terminals 5m. The master management apparatus 6 manages each of the local management apparatuses 7-r.

The configuration of the management apparatus 4 is described later. When the management apparatus 4 includes a plurality of computers, the components of the management apparatus 4 (described later) are dispersedly arranged in a plurality of computers. In addition, arbitrary components can be provided in two or more computers in a duplicated manner. With this, distributed processing and redundancy are implemented, for example.

Each of the examiner terminals (i.e., the internal examiner terminals 3-n and the external examiner terminals 5m) includes a computer for the use of an examiner who supervises and manages the examination being performed using any of the plurality of ophthalmic examination apparatus 2-iₙ.

### <Configuration of the Ophthalmic Examination Apparatus>

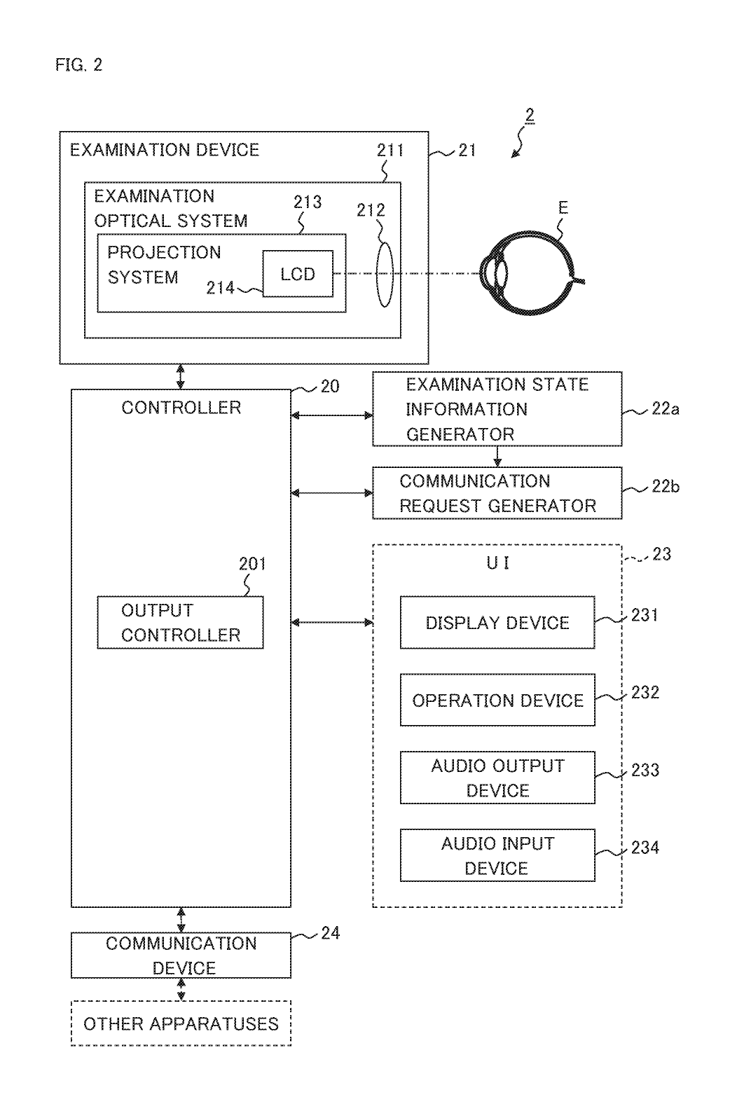

A description is given of an example of the configuration of the ophthalmic examination apparatus 2-iₙ. The ophthalmic examination apparatus 2 illustrated in FIG. 2 as an example corresponds to each of the plurality of ophthalmic examination apparatuses 2-iₙ illustrated in FIG. 1A. Similarly, each of the internal examiner terminal 3-n may sometimes be referred to simply as the “internal examiner terminal 3”, and each of the external examiner terminals 5m may sometimes be referred to simply as the “external examiner terminal 5”.

The ophthalmic examination apparatus 2 includes the controller 20, the examination device 21, the examination state information generator 22a, the communication request generator 22b, the user interface (UI) 23, and the communication device 24. These elements may be integrally provided (that is, may be provided in a single housing), or distributed in two or more housings. Examples of the former include a refractometer, a keratometer, a tonometer, a specular microscope, a wave front analyzer, a perimeter, a micro perimeter, an OCT apparatus, a fundus camera, an SLO, and the like. Examples of the latter include a visual acuity test apparatus that include a visual target presenting apparatus and a phoropter. Besides, a part or whole of the controller 20 may be implemented by a personal computer, a portable terminal, or the like. Further, a part or whole of the user interface 23 may be implemented by a personal computer, a portable terminal, a television receiver, a smart TV, or the like.

### <Controller 20>

The controller 20 performs various types of control and operations. The controller 20 includes one or more processors. The “processor” as used herein is a circuit such as, for example, a central processing unit (CPU), a graphics processing unit (GPU), an application specific integrated circuit (ASIC), and a programmable logic device (PLD). Examples of the PLD include a simple programmable logic device (SPLD), a complex programmable logic device (CPLD), and a field programmable gate array (FPGA). For example, the controller 20 reads a program stored in a memory circuit or a storage and executes the program, thereby implementing the functions of the present embodiment. The controller 20 may further include a RAM, a ROM, a hard disk drive, a solid state drive, and the like.

### <Output Controller 201>

The controller 20 includes the output controller 201. The output controller 201 performs control for outputting information sent from the internal examiner terminal 3 or the external examiner terminal 5 to the ophthalmic examination apparatus 2. Examples of the output information include visual information and audio information. The visual information is information recognized by the sensory system for vision, and includes text information, image information, and the like. The text information includes, for example, a text message that represents an instruction from the examiner. The image information includes, for example, a moving image of examiner's face and hand, and a mark, a sign or a symbol that represents an instruction from the examiner. The output controller 201 controls the liquid-crystal display (LCD) 214 and the display device 231 (described later) to output the visual information. The audio information is information recognized by the sensory system for hearing, and includes warning sound, a voice message, and the like. The output controller 201 controls the audio output device 233 (described later) to output the audio information. Specific examples of the processes performed by the output controller 201 are described later.

### <Examination Device 21>

The examination device 21 performs the examination of the eye under the control of the controller 20. The examination device 21 has a configuration corresponding to the type of the ophthalmic examination apparatus 2. The configuration of the examination device 21 may be at least a part of a known configuration or may include a known configuration. The examination device 21 includes an optical system to be described later, and mechanisms (not illustrated) such as an actuator, a power transmission mechanism, and the like.

When the examination device 21 has the function of measuring the properties of the subject's eye E, the examination device 21 includes at least an optical system for projecting light onto the subject's eye and may further include an optical system for detecting the returning light of the light projected onto the eye, for example. In addition, the examination device 21 may include optical systems for implementing various kinds of functions such as an optical system for projecting a fixation target onto the subject's eye, an optical system for performing alignment, and an optical system for performing focus adjustment. The examination device 21 may also include a processor for processing the detection result of the returning light such as image signals or video signals.

When the examination device 21 has the function of photographing the subject's eye E, the examination device 21 includes, for example, an optical system for projecting light onto the subject's eye and an optical system for detecting the returning light of the light projected onto the eye. As in the case of the property measurement function, the examination device 21 may include various kinds of optical systems. The examination device 21 may also include a processor for processing the detection result of the returning light such as image signals or video signals.

The optical systems as described above correspond to the examination optical system 211. The examination optical system 211 includes the optical element 212 and the projection system 213. The optical element 212 is applied to the subject's eye E to direct a light beam output from the projection system 213 to the subject's eye E. The optical element 212 may include any of various types of optical elements such as a lens (e.g., an objective lens), a prism (e.g., an objective prism), a concave mirror (e.g., a parabolic mirror), and a glass plate.

The projection system 213 projects the light beam for examining the subject's eye E (examination light beam) onto the subject's eye E through the optical element 212. The projection system 213 is provided with the LCD 214. The LCD 214 operates under the control of the controller 20. The LCD 214 has the function of displaying information used for examination (examination information such as a visual target and a fixation target) and the function of displaying information received from the internal examiner terminal 3 or the external examiner terminal 5. The latter is the function of an information presenting optical system. The LCD 214 corresponds to a display of the information presenting optical system. The projection system 213 also implements the function of a light guide system that guides a light beam (display light beam), which is output from the LCD 214 and corresponds to the information sent from the internal examiner terminal 3 or the external examiner terminal 5, to the subject's eye E through the optical element 212. In this way, the examination optical system and the information presenting optical system are common in the examination device 21 of the present embodiment. On the other hand, the examination optical system and the information presenting optical system may be configured separately, an example of which will be described later.

<Examination State Information Generator 22a>

The examination state information generator 22a generates examination state information indicating the state of the examination of the subject's eye E. The examination state information includes the progress state of examination (e.g., examination phase), on-going state (interim report) of examination (e.g., the history of visual targets having been presented in a visual acuity test), examination time (e.g., examination start time, elapsed time), or the like. When the ophthalmic examination apparatus 2 has the function of photographing the subject's eye E or the subject, the examination state information may include a captured image (e.g., a still image or a moving image) of the subject's eye E or the subject. The examination state information may include audio information of the subject input with the audio input device 234. The examination state information generated is, for example, sent to the internal examiner terminal 3 and/or the external examiner terminal 5, and is referred to by the examiner. In the present embodiment, the examination state information generated is also sent to the communication request generator 22b. The examination state information generator 22a includes, for example, a processor and a computer program for carrying out examination or a computer program for monitoring it.

The examination state information generator 22a need not necessarily process all the information sent from the ophthalmic examination apparatus 2 to the internal examiner terminal 3 and/or the external examiner terminal 5. For example, a moving image of the subject's eye E, the audio information of the subject, or the like, as it is, can be sent to the internal examiner terminal 3 and/or the external examiner terminal 5. With this, the states of the subject's eye E, the states of the subject, a request from the subject, or the like can be provided to the examiner with substantially no time lag.

<Communication Request Generator 22b>

The communication request generator 22b generates a request (communication request) for calling the internal examiner and/or the external examiner. The communication request generator 22b is configured to be capable of performing at least one of the following: processing of generating a communication request in response to an instruction from a subject; and processing of generating a communication request based on the examination state information generated by the examination state information generator 22a.

The instruction from the subject is input using the user interface 23. The instruction is input, for example, with the operation device 232 and/or the audio input device 234 (described later). More specifically, the subject operates the operation device 232 to perform a predetermined operation (e.g., pressing a button, or tilting a lever) in order to call an internal examiner and/or an external examiner. Further, the subject can input the request for calling by operating the software keys displayed on the display device 231 (described later) using the operation device 232. Alternatively, the subject can operate the software keys displayed on a touch panel display with a finger to input the request for calling. In addition, when the subject voices a message indicating that he or she has not perceived the contents of the examination or is in need of an instruction from the internal examiner and/or the external examiner, the audio information can be detected by the audio input device 234. According to the present example, it is possible to provide assistance in accordance with the subject's needs.

Described below is an example of the case for generating a communication request based on the examination state information. The examination state information generator 22a successively generates examination state information while monitoring the examination state. The communication request generator 22b determines whether or not the generated examination state information meets a predetermined condition. Examples of the predetermined condition include the followings: the examination is not proceeding; interim results (for example, the presentation history of the visual targets in the visual acuity test) are inappropriate; elapsed time from the start of the examination is long; eyelid is closed for a long time; the fixation of the subject's eye E is not applicable, and the like. Non-progression of the examination can be recognized, for example, by detecting the fact that the examination phase has not shifted for a predetermined time or more (for example, the fact that the same visual target is being presented). The inappropriateness of the interim results of the examination can be recognized, for example, by detecting the inappropriateness of the presentation order of the visual targets (for example, the contents of responses are not converging). Closed eyelids and fixation state of the eye E can be recognized by analyzing the photographed image of the eye E. Such image analysis includes, for example, the detection of the pupil. According to the present example, it is possible to automatically detect a situation that requires assistance.

The communication request generated by the communication request generator 22b is sent to the other apparatus (e.g., the management apparatus 4, the internal examiner terminal 3, the external examiner terminal 5) by the communication device 24.

### <User Interface 23>

The user interface 23 has the function of outputting information for the subject (and the internal examiner), and the function of inputting information and providing operating instructions. The user interface 23 includes the display device 231 and the audio output device 233 for the former function, and the operation device 232 and the audio input device 234 for the latter function.

The display device 231 includes a display device such as a flat panel display. The operation device 232 includes operation devices such as buttons, keys, a joystick, knobs, and an operation panel provided on the cabinet or outside of the ophthalmic examination apparatus 2. The operation device 232 may include operation devices (a mouse, a keyboard, a track pad, buttons, a touch panel, etc.) of a personal computer that is connected to the ophthalmic examination apparatus 2. The user interface 23 may include a device such as a touch panel that functions as both the display device 231 and the operation device 232, and a graphical user interface (GUI). The user interface 23 may include a computer program and a processor for implementing operation and input based on the subject's voice input from the audio input device 234.

The audio output device 233 includes, for example, a circuit for processing audio information (audio signals) to be output, and a speaker for outputting audio information processed by the circuit. The audio input device 234 includes a microphone for converting audio information into an electrical signal, and a circuit for processing the electrical signal.

### <Communication Device 24>

The communication device 24 executes data communication with another apparatus (e.g., the management apparatus 4, the internal examiner terminal 3, the external examiner terminal 5). The system of the data communication may be arbitrary. For example, the communication device 24 includes a communication interface conforming to the Internet, a communication interface conforming to LAN, a communication interface conforming to near field communication, and the like. The data communication may be either wireless communication or wired communication. Data sent and received by the communication device 24 may be encrypted. In this case, for example, the controller 20 includes an encryptor that encrypts data to be sent, and a decoder that decodes data having been received.

### <Configuration of the Internal Examiner Terminal>

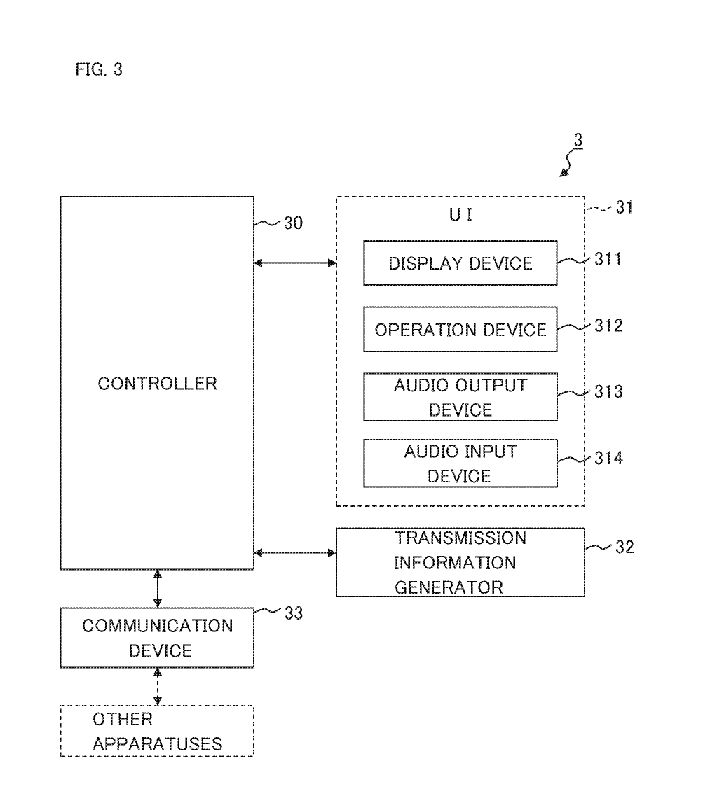

A description is given of an example of the configuration of the internal examiner terminal 3. The internal examiner terminal 3 illustrated in FIG. 3 as an example includes the controller 30, the user interface (UI) 31, the transmission information generator 32, and the communication device 33.

### <Controller 30>

The controller 30 controls each part of the internal examiner terminal 3 and performs various types of arithmetic operations. The controller 30 includes one or more processors. The controller 30 may further include a RAM, a ROM, a hard disk drive, a solid state drive, and the like.

### <User Interface 31>

The user interface 31 has the function of outputting information for a user, and the function for allowing the user to input information and provide operating instructions. As with the user interface 23 of the ophthalmic examination apparatus 2, the user interface 31 includes the display device 311, the operation device 312, the audio output device 313, and the audio input device 314.

For example, the controller 30 controls the display device 311 to display a screen (e.g., GUI) to present the examination state information successively sent from the ophthalmic examination apparatus 2 and to enter an instruction to the subject. In addition, the controller 30 controls the audio output device 313 to output audio information of the subject. With reference to the display information and audio information, the examiner can determine the content of an instruction, and enter the instruction using the operation device 312. Meanwhile, a voice instruction is entered through the audio input device 314.

Further, for example, the controller 30 controls the display device 311 to display a screen (e.g., GUI) to present information sent from another examiner terminal (e.g., other internal examiner terminal 3 or external examiner terminal 5) and to enter information to be sent to another examiner terminal. In addition, the controller 30 controls the audio output device 313 to output audio information that has been input to another examiner terminal. With reference to the display information and audio information sent from another examiner terminal, the examiner can determine a message to the subject or another examiner, and enter the message using the operation device 312. Meanwhile, a voice message is entered through the audio input device 314.

### <Transmission Information Generator 32>

Based on the information that the examiner has entered using the user interface 31, the transmission information generator 32 generates information (transmission information) for the user of another apparatus (e.g., the subject who uses the ophthalmic examination apparatus 2, the examiner who uses another examiner terminal). The transmission information generator 32 includes, for example, a processor and a transmission information generating program to be executed in conjunction with the screen (GUI) described above.

Note that the transmission information generator 32 need not necessarily process all the information sent from the internal examiner terminal 3 to another apparatus. For example, the moving image of the examiner, the audio information, or the like, as it is, can be sent to another apparatus. With this, a message from the examiner can be provide to the subject or another examiner with substantially no time lag.

### <Communication Device 33>

The communication device 33 performs data communication with another apparatus (e.g., the ophthalmic examination apparatus 2, the management apparatus 4, the external examiner terminal 5). The system of the data communication and encryption executed by the communication device 33 may be the same as those performed by the communication device 24 of the ophthalmic examination apparatus 2.

### <Configuration of the External Examiner Terminal>

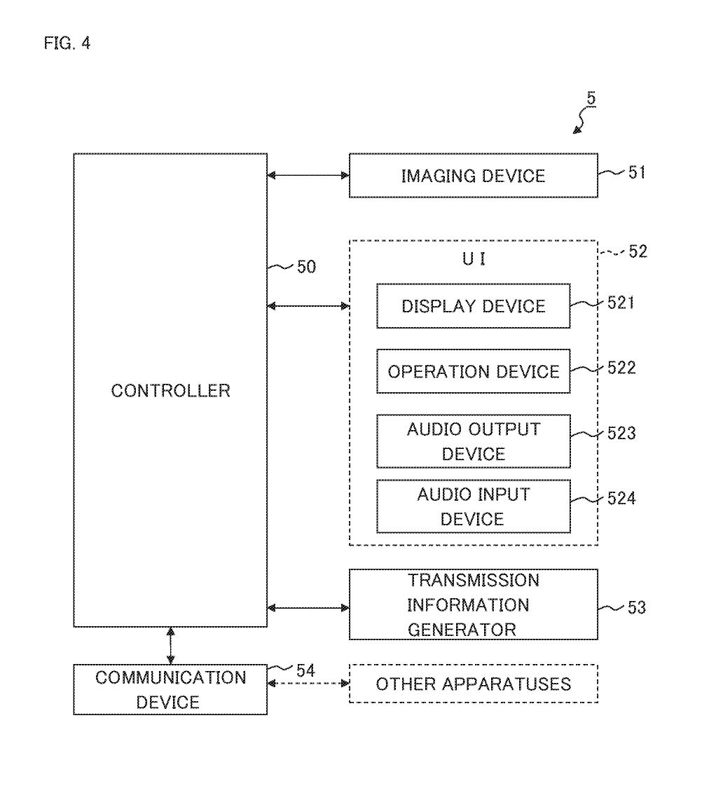

A description is given of an example of the configuration of the external examiner terminal 5. The external examiner terminal 5 illustrated in FIG. 4 as an example includes the controller 50, the imaging device 51, the user interface (UI) 52, the transmission information generator 53, and the communication device 54.

### <Controller 50>

The controller 50 controls each part of the external examiner terminal 5 and performs various types of arithmetic operations. The controller 50 includes one or more processors. The controller 50 may further include a RAM, a ROM, a hard disk drive, a solid state drive, and the like.

### <Imaging Device 51>

The imaging device 51 is used to capture a moving image of the examiner, and includes, for example, a video camera. The object to be photographed includes a body part(s) (e.g., face, upper body, hand) used to provide an instruction to the subject.

### <User Interface 52>

The user interface 52 has the function of outputting information for the examiner, and the function for allowing the examiner to input information and provide operating instructions. As with the user interface 23 of the ophthalmic examination apparatus 2, the user interface 52 includes the display device 521, the operation device 522, the audio output device 523, and the audio input device 524.

For example, the controller 50 controls the display device 521 to display a screen (e.g., GUI) to present the examination state information successively sent from the ophthalmic examination apparatus 2 and to enter an instruction to the subject. In addition, the controller 50 controls the audio output device 523 to output audio information of the subject. With reference to the display information and audio information, the examiner can determine the content of an instruction, and enter the instruction using the operation device 522. Meanwhile, a voice instruction is entered through the audio input device 524.

Further, for example, the controller 50 controls the display device 521 to display a screen (e.g., GUI) to present information sent from another examiner terminal (e.g., the internal examiner terminal 3, another external examiner terminal 5) and to enter information to be sent to another examiner terminal. In addition, the controller 50 controls the audio output device 523 to output audio information that has been input to another examiner terminal. With reference to the display information and audio information sent from another examiner terminal, the examiner can determine the message to the subject or another examiner, and enter the message using the operation device 522. Meanwhile, a voice message is entered through the audio input device 524.

### <Transmission Information Generator 53>

Based on the information that the examiner has entered using the user interface 52, the transmission information generator 53 generates information (transmission information) for the user of another apparatus (e.g., the subject who uses the ophthalmic examination apparatus 2, the examiner who uses another examiner terminal). The transmission information generator 53 includes, for example, a processor and a transmission information generating program to be executed in conjunction with the screen (GUI) described above.

Note that the transmission information generator 53 need not necessarily process all the information sent from the external examiner terminal 5 to another apparatus. For example, the moving image of the examiner, the audio information, or the like, as it is, can be sent to another apparatus. With this, a message from the examiner can be provide to the subject with substantially no time lag.

### <Communication Device 54>

The communication device 54 performs data communication with another apparatus (e.g., the ophthalmic examination apparatus 2, the internal examiner terminal 3, the management apparatus 4). The system of the data communication and encryption performed by the communication device 54 may be the same as those performed by the communication device 24 of the ophthalmic examination apparatus 2.

### <Configuration of the Management Apparatus>

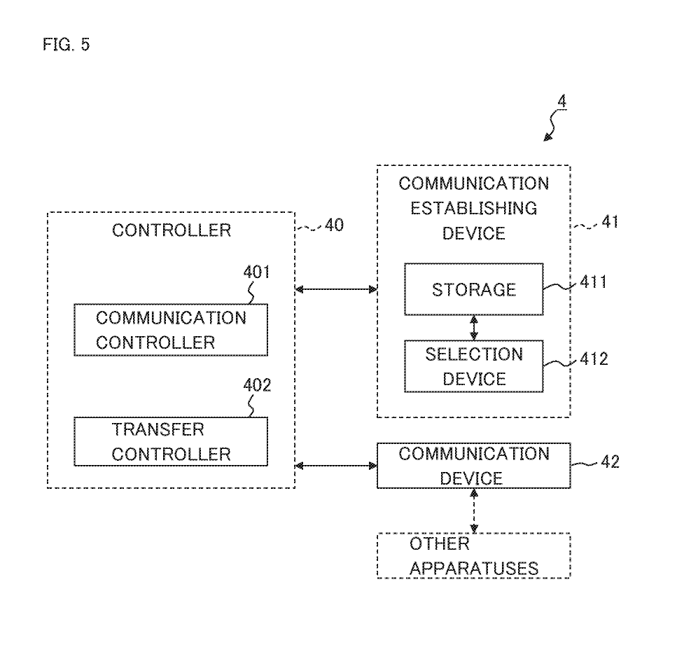

A description is given of an example of the configuration of the management apparatus 4. The management apparatus 4 illustrated in FIG. 5 includes the controller 40, the communication establishing device 41, and the communication device 42. At least one of these components is implemented, for example, by cooperation of hardware and software included in the master management apparatus 6 and/or the local management apparatus 7-r illustrated in FIG. 1B.

### <Controller 40>

The controller 40 controls each part of the management apparatus 4. For example, a part of the controller 40 (master controller) is provided in the master management apparatus 6, and the master controller performs control of each local management apparatus 7-r. The controller 40 may be capable of other kinds of arithmetic operations. The controller 40 includes one or more processors. The controller 40 may further include a RAM, a ROM, a hard disk drive, a solid state drive, and the like.

The controller 40 includes the communication controller 401 and the transfer controller 402.

The communication controller 401 performs control related to the establishment of communication between the followings: between the ophthalmic examination apparatus 2 and the internal examiner terminal 3; between the ophthalmic examination apparatus 2 and the external examiner terminal 5; between the internal examiner terminal 3 and the external examiner terminal 5; between two or more ophthalmic examination apparatuses 2; between two or more internal examiner terminals 3; and between two or more external examiner terminal 5. For example, the communication controller 401 can send a control signal for establishing communication to each of the apparatuses selected from a plurality of apparatuses including the plurality of ophthalmic examination apparatuses 2, one or more internal examiner terminals 3, and one or more external examiner terminals 5. The selection of apparatuses is executed by the selection device 412 described later.

The transfer controller 402 performs controls related to the exchange of information between the apparatuses whose communication has been established by the communication establishing device 41 (and the communication controller 401). For example, the transfer controller 402 transfers the information sent from one of at least three apparatuses whose communication has been established by the communication establishing device 41 (and the communication controller 401), to at least one of the other two or more apparatuses from among the at least three apparatuses.

A specific example of processing executed by the transfer controller 402 will be described. When communication between the ophthalmic examination apparatus 2 and the external examiner terminal 5 is established, the transfer controller 402 transfers the information sent from the ophthalmic examination apparatus 2 (or the external examiner terminal 5) to the external examiner terminal 5 (or the ophthalmic examination apparatus 2). At this time, the transfer controller 402 may transmit information obtained by processing the information sent from the ophthalmic examination apparatus 2 etc. to the external examiner terminal 5 etc. For example, the transfer controller 402 can extract a part of the information transmitted from the ophthalmic examination apparatus 2 etc. and transmit the extracted information to the external examiner terminal 5 etc. Further, the information transmitted from the ophthalmic examination apparatus 2 etc. (e.g., image data of the subject's eye E) may be analyzed by the management apparatus 4 or another apparatus and the result obtained by the analysis (and original information) may be sent to the external examiner terminal 5 etc. Incidentally, the kinds of the processing of information are not limited to such extraction and analysis but may include data processing of an arbitrary type.

Another specific example of processing executed by the transfer controller 402 will be described. When communication between two examiner terminals is established, the transfer controller 402 transfers the information sent from one examiner terminal to another examiner terminal. At this time, the transfer controller 402 may transmit information obtained by processing the information sent from one examiner terminal to another examiner terminal. For example, the transfer controller 402 can extract a part of the information transmitted from one examiner terminal and send the extracted information to another examiner terminal. Further, the information transmitted from one examiner terminal (e.g., image data obtained by the imaging device 51) may be analyzed by the management apparatus 4 or another apparatus and the result obtained by the analysis (and original information) may be sent to another examiner terminal. Incidentally, the processing of information may include data processing of an arbitrary type.

### <Communication Establishing Device 41>

The communication establishing device 41 can perform processing for establishing communication between at least three apparatuses including at least one ophthalmic examination apparatus 2 and at least two examiner terminals. In the present embodiment, “establishment of communication” refers to a concept which includes, for example, at least one of the followings: (1) establishing unidirectional communication from a state in which communication is disconnected; (2) establishing bidirectional communication from a state in which communication is disconnected; (3) switching from a state in which only reception is possible to a state in which transmission is also possible; and (4) switching from a state in which only transmission is possible to a state in which reception is also possible.

As a specific example, “establishment of communication” may include switching from a state in which the examiner uses the internal examiner terminal 3 or the external examiner terminal 5 to monitor the examination being performed by using the ophthalmic examination apparatus 2 (i.e., a state in which only reception is possible) to a state in which sending instructions to the subject is possible using the internal examiner terminal 3 or the external examiner terminal 5. As another specific example, “establishment of communication” may include switching from a state in which one examiner uses one examiner terminal to monitor the state of examination assistance being performed by using another examiner terminal to a state in which sending messages to the another examiner terminal is possible from the one examiner terminal.

Further, the communication establishing device 41 can perform processing of disconnecting the established communication. In the present embodiment, “disconnecting communication” refers to a concept which includes, for example, at least one of the followings: (1) disconnecting communication from a state in which unidirectional communication has been established; (2) disconnecting communication from a state in which bidirectional communication has been established; (3) switching from a state in which bidirectional communication has been established to unidirectional communication; (4) switching from a state in which transmission and reception are possible to a state in which only reception is possible; and (5) switching from a state in which transmission and reception are possible to a state in which only transmission is possible.

As a specific example, “disconnecting communication” may include switching from a state in which the external examiner is able to send instructions to the subject using the external examiner terminal 5 (i.e., a state in which both transmission and reception are possible) to a state in which only the monitoring of the examination is possible using the external examiner terminal 5 (i.e., a state in which only reception is possible). As another specific example, “disconnecting communication” may include switching the communication state between two examiner terminals from bidirectional communication to unidirectional communication or to no communication.

Each of the ophthalmic examination apparatuses 2 and the examiner terminals (i.e., the internal examiner terminals 3 and the external examiner terminals 5) can send a communication request (call request) for calling another apparatus (the user thereof) to another apparatus. In addition, each of the ophthalmic examination apparatuses 2 and the examiner terminals (i.e., the internal examiner terminal 3 and the external examiner terminal 5) can send a communication request (interruption request) for interrupting communication between other two apparatuses to other apparatuses. Each of the ophthalmic examination apparatuses 2 and the examiner terminals (i.e., the internal examiner terminals 3 and the external examiner terminals 5) may be capable of sending another type of communication request. The communication request is sent manually or automatically.

In the present example, each of the ophthalmic examination apparatuses 2 and the examiner terminals (i.e., the internal examiner terminals 3 and the external examiner terminals 5) can send a communication request to the management apparatus 4. The management apparatus 4 (the communication device 42 thereof) receives the communication request sent from the ophthalmic examination apparatus 2 or the examiner terminal, and executes processing according to the communication request.

In the present embodiment, the communication establishing device 41 may include the selection device 412. Based on the received communication request, the selection device 412 selects two or more apparatuses, other than the apparatus that has sent the communication request, from the ophthalmic examination apparatuses 2, the internal examiner terminals 3, and the external examiner terminals 5.

For example, upon receiving a communication request from the ophthalmic examination apparatus 2, the selection device 412 selects, for example, two or more examiner terminals including the internal examiner terminal 3 used in the facility in which the same ophthalmic examination apparatus 2 is installed and any of the external examiner terminals 5. The communication establishing device 41 establishes communication between the selected two or more examiner terminals and the ophthalmic examination apparatus 2 which has sent the communication request. When bidirectional communication or the like between the ophthalmic examination apparatus 2 and the internal examiner terminal 3 has already been established, the communication establishing device 41 may only execute establishing of the communication between, for example, an examiner terminal other than the same internal examiner terminal 3 (e.g., the internal examiner terminal 3 installed in the same facility or another facility, the external examiner terminal 5) and the ophthalmic examination apparatus 2. By such communication establishment processing, it becomes possible to automatically select an examiner in accordance with a request of the subject of the ophthalmic examination apparatus 2 and provide support for the examination.

Another example will be described. Upon receiving a communication request from the examiner terminal (e.g., the internal examiner terminal 3 or the external examiner terminal 5), the selection device 412 selects, for example, two or more apparatuses including the ophthalmic examination apparatus 2 installed in the facility in which the same internal examiner terminal 3 is installed and any of the external examiner terminals 5. The communication establishing device 41 establishes communication between the selected one or more ophthalmic examination apparatuses 2, the selected one or more examiner terminals, and the examiner terminal which has sent the communication request. For example, when bidirectional communication or the like between the examiner terminal which has sent the communication request and the ophthalmic examination apparatus 2 has already been established, the communication establishing device 41 may only execute establishing of the communication between an examiner terminal other than the same examiner terminal and the ophthalmic examination apparatus 2. By such communication establishment processing, it becomes possible to automatically select another examiner or a subject in accordance with a request from the examiner and provide support for the examination.

The selection of an apparatus in accordance with a communication request can be performed, for example, based on preset attributes. Examples of the attributes include the type of examination, the degree of expertise and the level of skill required for assisting the examination, the type of language, and the like. In order to realize the present example, the communication establishing device 41 may include the storage 411 in which the attribute information prepared in advance is stored.

In the present example, the attribute information includes information indicating the attributes of the internal examiner terminal 3 and/or its user (i.e., internal examiner) and information indicating the attributes of the external examiner terminal 5 and/or its user (i.e., external examiner). Here, the internal examiner and the external examiner are identified using, for example, a pre-assigned examiner identification. Further, the internal examiner terminal 3 and the external examiner terminal 5 are identified using, for example, a pre-assigned apparatus identification or network address. In a typical example, the attribute information includes the attributes of each examiner (each of the internal examiner and the external examiner) such as the type of the examination that the examiner can support, the examiner's degree of expertise and the level of skill, and the type of language the examiner is able to use.

In another example, the attribute information may include one of the followings: information indicating the attributes of the internal examiner terminal 3 and/or its user (i.e., internal examiner); and information indicating the attributes of the external examiner terminal 5 and/or its user (i.e., external examiner). Alternatively, the attribute information may include information indicating the attributes of a predetermined examiner terminal or its user (i.e., examiner) irrespective of the type of the examiner terminal. The information included in the attribute information (e.g., information on the examiner terminal, the examiner, or attributes) may be edited.

When the selection device 412 refers to the attribute information, the communication request transmitted from the ophthalmic examination apparatus 2, the internal examiner terminal 3, or the external examiner terminal 5 may include information related to the attributes. For example, the communication request (e.g., the call request) transmitted from the ophthalmic examination apparatus 2 may include any of the followings: (1) information indicating the type of examination being performed by the ophthalmic examination apparatus 2; (2) information indicating the degree of difficulty of supporting the examination (e.g., information indicating the fact that the subject's eye or the subject is in difficulty of undergoing the examination, or information indicating the progress state of the examination); (3) information indicating the name of disease, age, etc. of the subject; and (4) information indicating a language(s) the subject uses. The selection device 412 selects at least one from any of the internal examiner terminals 3 and any of the external terminals 5, based on the attribute information and the communication request received. At this time, the selection device 412 collates the information related to the attributes included in the communication request with the information included in the attribute information. With this, the selection device 412 selects, for example, the internal examiner terminal 3 and/or the external examiner terminal 5 corresponding to any one of the following examiners: (1) an examiner who is capable of supporting the concerned type of examination; (2) an examiner who is capable of supporting the concerned level of difficulty of the examination; (3) an examiner who is capable of supporting the subject of the concerned disease or the concerned age; and (4) an examiner who is capable of using the concerned language. Here, the correspondence between the internal examiner and the internal examiner terminal 3, and the correspondence between the external examiner and the external examiner terminal 5 can be made by, for example, referring to the examiner ID input, at the time of logging in, to the examiner terminal to which the apparatus ID has been assigned.

### <Communication Device 42>

The communication device 42 performs data communication with another apparatus (e.g., the ophthalmic examination apparatus 2, the internal examiner terminal 3, the external examiner terminal 5). The system of the data communication and encryption may be performed in the same manner as in the communication device 24 of the ophthalmic examination apparatus 2.

### <Usage Mode>

A description is given of a typical example of the usage mode of the ophthalmic examination system 1 of the present embodiment.

### <First Example of Usage Mode>

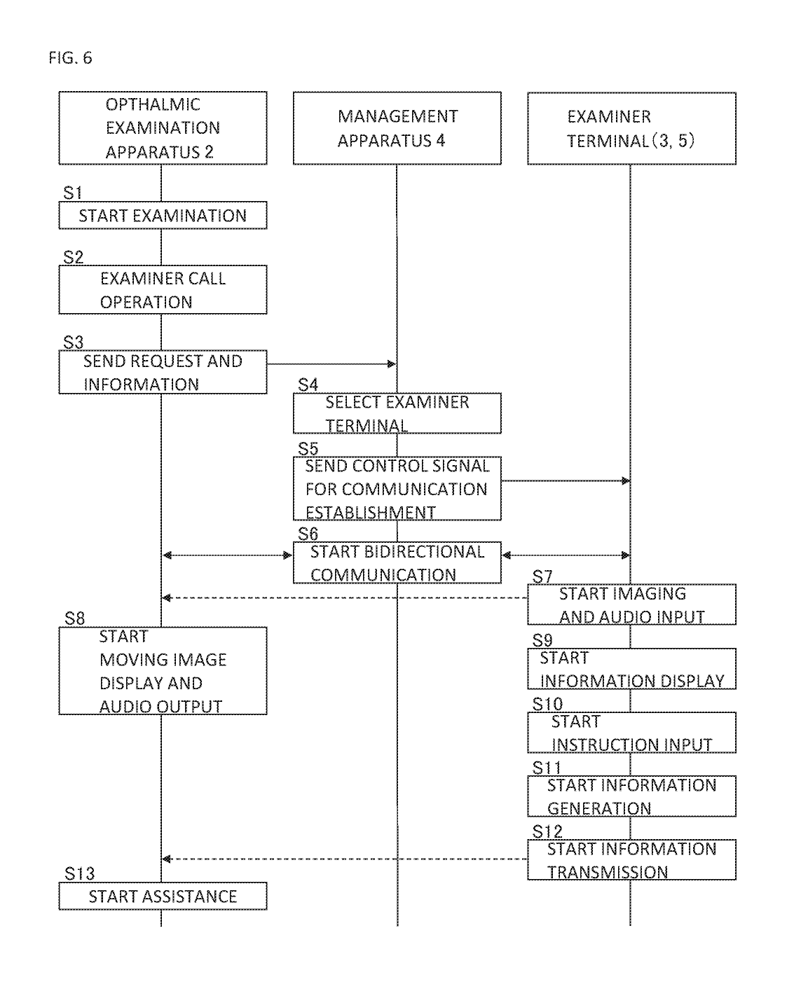

A description is given of the first example of the usage mode of the ophthalmic examination system 1. In the present example, a subject makes a request for an examiner's assistance. In the present example, reference is made to FIG. 6.

### (S1: Start Examination)

Examination of the subject's eye E using the ophthalmic examination apparatus 2 which is one of the plurality of ophthalmic examination apparatus 2-iₙ starts. The communication (e.g., unidirectional communication or bidirectional communication) between the ophthalmic examination apparatus 2 and one or more examiner terminals (i.e., the internal examiner terminal 3 and/or the external examiner terminal 5) may be started in this stage. In response to the commencement of the examination, the controller 20 of the ophthalmic examination apparatus 2 controls the examination state information generator 22a to start generating the examination state information.

### (S2: Examiner Call Operation)

When the subject needs assistance from the examiner, the subject calls the examiner who uses the user interface 23. Examples of the case where the assistance of the examiner is required include a case where the subject does not know how to conduct the examination, when he or she is unable to understand the language used, when he or she has become tired, and when he or she wants to return (or redo) the steps of the examination. Examples of the way how the user uses the user interface 23 in order to input the request for calling include performance of a predetermined operation with the operation device 232, and input of voice with the audio input device 234.

### (S3: Send Request and Information)

The controller 20 controls the communication device 24 to send the examiner call request (that is, the communication request) input in step S2 to the management apparatus 4 together with prescribed information. Examples of the information sent with the examiner call request include identification information assigned in advance to the ophthalmic examination apparatus 2 (i.e., the apparatus ID), identification information assigned in advance to the subject (i.e., the subject ID), identification information assigned in advance to the facility where the ophthalmic examination apparatus 2 is installed (i.e., the facility ID), at least part of the examination state information generated to that point, and the content of the examiner call request.

### (S4: Select Examiner Terminal)

The management apparatus 4 receives the examiner call request and the information sent from the ophthalmic examination apparatus 2 in step S3 by the communication device 42. The selection device 412 selects an examiner to assist the subject. More specifically, the selection device 412 selects at least two of the following apparatuses: the internal examiner terminal 3 installed in the facility where the subject is undergoing examination; the internal examiner terminals 3 installed in other facilities; and the external examiner terminals 5.

In a typical example, communication between the internal examiner terminal 3 installed in the facility where the subject is undergoing examination and the ophthalmic examination apparatus 2 has already been started at the stage of step S1. At the stage of step S4 in a typical example, one or more examiner terminals different from the same internal examiner terminal 3 (e.g., an internal examiner terminal(s) 3 installed in the same facility or installed in another facility, and/or, an external examiner terminal(s) 5) are selected. In other words, in the present example, the selection device 412 selects two or more examiner terminals including the internal examiner terminal 3 whose communication has already started and one or more other examiner terminals.

A description is given of an example of processing for selecting an examiner terminal.

The selection device 412 may have, for example, the function of monitoring the operating state (e.g., communication establishment information) of each examiner terminal (i.e., the internal examiner terminals 3 and the external examiner terminals 5). With this monitoring function, the selection device 412 can select any of the examiner terminals that are not currently running (e.g., the examiners who are not currently performing assistance of subjects, the examiners who are currently performing assistance of subjects yet still available for providing assistance of other subjects, the examiners who do not have a reservation for assistance of subjects). Thereby, it is possible to assign an examiner who is not currently assisting other subjects or the like, to the assistance of the subject who has sent the call request.

In addition, the monitoring function can be implemented, for example, by managing the operating states of each examiner terminal using a flag or the like, based on information input from the examiner terminals periodically or non-periodically, reactions to information sent to the examiner terminals, or the like.

In another example, the selection device 412 can determine whether or not the communication has been actually established for the examiner terminal corresponding to the communication request among the plurality of examiner terminals including the internal examiner terminals 3 and the external terminals 5. If the communication of the same examiner terminal has not been established, the selection device 412 can establish the communication between the same examiner terminal and the ophthalmic examination apparatus 2. On the other hand, if the communication of the same examiner terminal has already been established, the selection device 412 can establish the communication between another examiner terminal and the ophthalmic examination apparatus 2.

A specific example will be described. When all the internal examiner terminals 3 in the facility are currently running, that is, when the communication between each of the internal examiner terminals 3 in the facility and another ophthalmic examination apparatus 2 and/or the external examiner terminal 5 has been established, the selection device 412 can select any of the non-active examiner terminals among the internal examiner terminals 3 installed in other facilities and/or the external examiner terminals 5, for example.

On the other hand, when there exist internal examiner terminals 3 in the facility that are not running, the selection device 412 can select any one of these non-active internal examiner terminals 3.

With the present example, it is possible to provide a prompt assistance to the subject by comprehensively taking into account the operating states of a plurality of examiner terminals. Incidentally, the present example can also be implemented, for example, by managing the operating states of each examiner terminal using a flag or the like.

### (S5: Send Control Signal for Communication Establishment)

To each of the examiner terminals selected in step S4, the communication controller 401 sends a control signal for establishing the communication between the examiner terminal and the ophthalmic examination apparatus 2. In the example illustrated in FIG. 6, the examiner terminal may be the internal examiner terminal 3 or the external examiner terminal 5.

### (S6: Start Bidirectional Communication)

By the control signal sent in step S5, bidirectional communication between the examiner terminal and the ophthalmic examination apparatus 2 is established. At this time, it is also possible to further establish the communication between two or more examiner terminals. As described above, the communication may be established in the previous stage.

### (S7: Start Imaging and Audio Input)

When the communication between the ophthalmic examination apparatus 2 and the external examiner terminal 5 has been established in step S5, the transfer controller 402 of the management apparatus 4 sends the information sent from the ophthalmic examination apparatus 2 in step S3, to this external examiner terminal 5. The controller 50 of the external examiner terminal 5 controls the imaging device 51 to start capturing a moving image of the examiner. At this time, input of voice coming out of the examiner is also started. The frames successively acquired by the imaging device 51 are transferred to the ophthalmic examination apparatus 2 in real time via the management apparatus 4. This transfer process is performed by the transfer controller 402.

In case where the communication between the ophthalmic examination apparatus 2 and the internal examiner terminal 3 has been established in step S5 as well, similar imaging can be performed. For example, the same processing can be executed when the communication has been established between the ophthalmic examination apparatus 2 and the internal examiner terminal 3 installed in another facility wherein this internal examiner terminal 3 has the imaging device that has the same configuration as the imaging device 51 shown in FIG. 4. In a specific example, the transfer controller 402 of the management apparatus 4 sends the information sent from the ophthalmic examination apparatus 2 in step S3, to the internal examiner terminal 3. The controller 30 of the internal examiner terminal 3 controls the imaging device (not shown) to start capturing a moving image of the examiner. At this time, input of voice coming out of the examiner is also started. The frames successively acquired by the imaging device are transferred to the ophthalmic examination apparatus 2 in real time via the management apparatus 4. This transfer process is performed by the transfer controller 402.

Also in case where the communication has been established between the two or more examiner terminals selected in step S4, similar imaging can be performed. For example, the same processing can be executed when the communication has been established between the internal examiner terminal 3 installed in the same facility as the ophthalmic examination apparatus 2 and the external examiner terminal 5 (or the internal examiner terminal 3 installed in another facility). In a specific example, the controller 50 of the external examiner terminal 5 controls the imaging device 51 to start capturing a moving image of the examiner. At this time, input of voice coming out of the examiner is also started. The frames successively acquired by the imaging device 51 are transferred to the internal examiner terminal 3 in real time via the management apparatus 4. This transfer process is performed by the transfer controller 402.

### (S8: Start Moving Image Display and Audio Output)

The ophthalmic examination apparatus 2 receives the frames and audio information successively sent from the external examiner terminal 5 (and/or those from the internal examiner terminal 3). The output controller 201 displays the successively received frames in real time on the LCD 214. Thus, a moving image of the examiner is displayed in real time. The output controller 201 also controls the audio output device 233 to output the audio information in real time. Thereby, the voice of the examiner is output in real time.

Through such cooperative operations as above, the subject can perceive the face, gestures, voice, and the like of the examiner in real time. On the other hand, the examiner can also perceive the voice, operations, and the like of the subject in real time. Such bidirectional information exchange is implemented mainly thanks to the transfer controller 402 of the management apparatus 4.

Likewise, when the communication between the two or more examiner terminals selected in step S4 has been established, one examiner terminal of the two or more examiner terminals receives the frames and audio information successively sent from another examiner terminal. In a typical example, the “one examiner terminal” is the internal examiner terminal 3 installed in the same facility as the ophthalmic examination apparatus 2, and the “another examiner terminal” is the external examiner terminal 5 or the internal examiner terminal 3 installed in another facility. If this is the case, the controller 30 of the internal examiner terminal 3 installed in the same facility as the ophthalmic examination apparatus 2 displays the frames successively received from the external examiner terminal 5 (or, from the internal examiner terminal 3 installed in another facility) in real time on the display device 311. With this, the moving image of the examiner who uses the external examiner terminal 5 (or the internal examiner terminal 3 installed in another facility) is displayed in real time. In addition, the controller 30 controls the audio output device 313 to output the audio information successively received from the external examiner terminal 5 (or, from the internal examiner terminal 3 installed in another facility) in real time. With this, the voice of the examiner who uses the external examiner terminal 5 (or the internal examiner terminal 3 installed in another facility) is output in real time.

Through such cooperative operations as above, it is possible to provide real-time support to the examiner who wishes to receive support from another examiner. Such bidirectional information exchange is implemented mainly thanks to the transfer controller 402 of the management apparatus 4.

### (S9: Start Information Display)

In order to facilitate the assist work by the examiner, the controller (e.g., the controller 30 or 50) of the examiner terminal (e.g., the internal examiner terminal 3 or the external examiner terminal 5) displays the screen (e.g., GUI) described above on the display device (e.g., the display device 311 or 521). In addition, the controller (e.g., the controller 30 or 50) of the examiner terminal (e.g., the internal examiner terminal 3 or the external examiner terminal 5) can display the information transferred from the ophthalmic examination apparatus 2 via the management apparatus 4 (especially, the examination state information) on the screen. Upon receipt of new examination state information, the controller (e.g., the controller 30 or 50) updates the display contents. Thereby, the examiner can perceive the states of the examination currently performed using the ophthalmic examination apparatus 2 substantially in real time, and provide appropriate assistance at an appropriate timing by using the visual information and the audio information.

### (S10: Start Instruction Input)

The examiner enters a desired instruction using the operation device (e.g., the operation devices 312 or 522), and the GUI etc. Examples of the contents of the instruction include the change of examination, the presentation of text information, and the presentation of image information. Besides, examples of instructions issued using a means other than the GUI include instructions by body language, instructions by voice, and the like.

### (S11: Start Information Generation)

Based on the contents (instruction) entered in step S10, the transmission information generator 53 of the external examiner terminal 5 generates transmission information for the subject. The transmission information is generated, for example, each time the examiner enters an instruction.

In addition, the transmission information generator 32 of the internal examiner terminal 3 generates the transmission information for the subject, based on the contents (instruction) entered in step S10. The generation of transmission information is executed, for example, each time the examiner enters an instruction.

When the communication has been established between the two or more examiner terminals selected in step S4, the transmission information generator 53 of the external examiner terminal 5 can generate transmission information for another examiner terminal. Similarly, the transmission information generator 32 of the internal examiner terminal 3 can generate transmission information for another examiner terminal.

### (S12: Start Information Transmission)

The examiner terminal (e.g., the external examiner terminal 5 or the internal examiner terminal 3) sends the transmission information generated in step S11 to the ophthalmic examination apparatus 2 via the management apparatus 4. This processing is performed by the transfer controller 402.

Similarly, when the communication between the two or more examiner terminals selected in step S4 has been established, one examiner terminal sends the transmission information generated in step S11 to another examiner terminal via the management apparatus 4. This processing is performed by the transfer controller 402.

### (S13: Start Assistance)

The ophthalmic examination apparatus 2 receives the transmission information sent from the examiner terminal in step S12. The output controller 201 displays the moving image and the instruction from the examiner on the LCD 214 based on the transmission information.

Similarly, when the transmission information has been sent from one examiner terminal to another examiner terminal in step S12, the another examiner terminal receives the transmission information sent from the one examiner terminal, and displays a message based on the transmission information.

Described below is a specific example of the instruction provided from the examiner to the subject. Here, it is assumed that the change of examination includes the operation of switching the types of the visual targets used in the visual acuity test from a Landolt ring to an illustration chart. The illustration chart is a visual target made of an illustration. More specifically the illustration chart is, for example, a visual target showing a familiar scenery in which characters and images corresponding to several levels of visual acuity scores. An instruction to change examination is issued by, for example, operating a predetermined software key provided in the GUI. Having received the instruction to change the examination, the output controller 201 of the ophthalmic examination apparatus 2 switches the visual target for the visual acuity test displayed on the LCD 214 from a Landolt ring to the illustration chart.

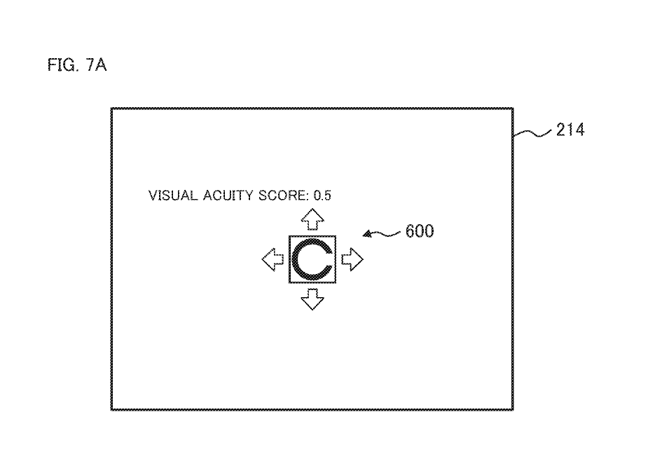

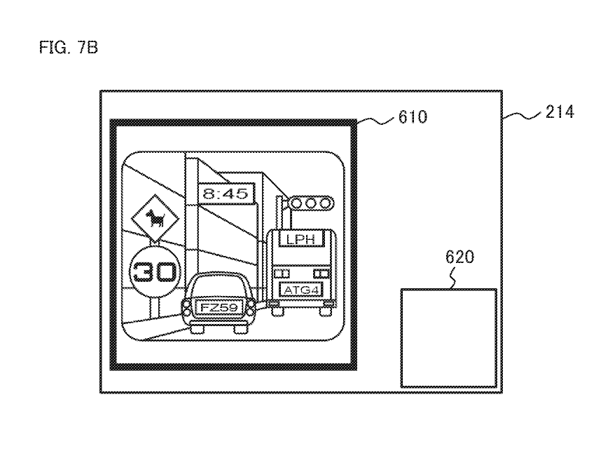

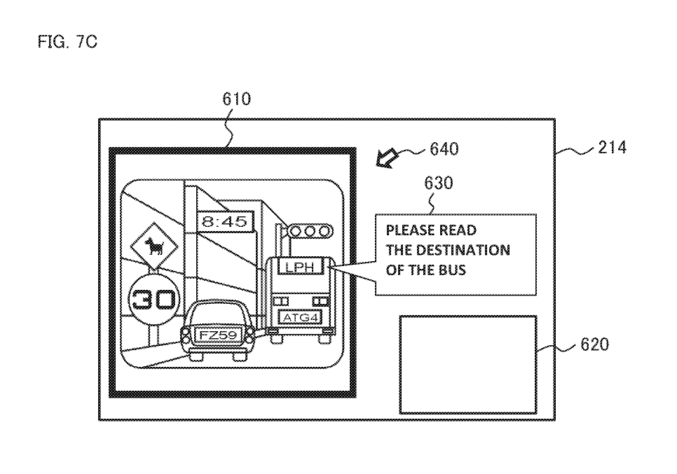

FIG. 7A illustrates an example of display for visual acuity test using a Landolt ring. As illustrated in FIG. 7A, the display content 600 includes the Landolt ring in a “C” shape, a character string “visual acuity: 0.5” representing the visual acuity score corresponding to the Landolt ring, and arrow images pointing up, down, left, and right directions, respectively. The arrow images are arranged around the Landolt ring to enter the selection of the direction of the cutout in the Landolt ring.

FIG. 7B illustrates an example of display for visual acuity test using an illustration chart. In the example of FIG. 7B, the illustration chart 610 is displayed on the LCD 214. The illustration chart 610 shows an ordinary motor vehicle and a bus running on the road, a road sign, a digital clock, and so on. The speed limit “30” indicated by the road sign, the time “8:45” indicated by the digital clock, the character string “LPH” that represents the destination of the bus, the character string “ATG4” written on the license plate of the bus, the character string “FZ59” written on the license plate of the ordinary motor vehicle are in their respective sizes corresponding to different visual acuity scores. As illustrated in FIG. 7B, the examiner image display area 620 is also displayed. In the examiner image display area 620, the moving image of the examiner being acquired by the examiner terminal is displayed in real time by the output controller 201.

FIG. 7C illustrates an example of display of an instruction from the examiner. For example, in order to make the subject read the character string “LPH” indicating the destination of the bus, the examiner performs an operation for that purpose using the GUI. The output controller 201 displays the instruction information 630 that indicates the content of instruction, based on the transmission information sent from the examiner terminal. The instruction information 630 of the present example is a balloon image in which the character string “please read the destination of the bus” is displayed. The balloon image points to the display of the destination of the bus. The examiner can also provide a voice instruction while displaying the instruction information 630.

If the information about the visual acuity of the subject's eye E (e.g., visual acuity score, refraction degree) is acquired in advance, the output controller 201 can adjust the display size of the instruction on the basis of this information. The output controller 201 stores, in advance, table information in which visual acuity scores and/or refraction degrees are associated with display sizes of character strings and/or display sizes of images. When the contents of the instruction include text information and/or image information, the output controller 201 selects the display size associated with the visual acuity score and/or the refractive power of the subject's eye E. Then, the output controller 201 displays the text information and/or the image information in the selected display size on the LCD 214.

In the example of FIG. 7C, the pointer 640 is also displayed. The examiner can move the pointer 640 in real time using the GUI. With this, the examiner can point at the position of the destination display of the bus, for example.

An example of processing that can be executed when the communication between the two or more examiner terminals selected in step S4 has been established will be described. In this case, it is possible to exchange messages between the two or more examiner terminals. In a typical example, the examiner, who uses the external examiner terminal 5 or an internal examiner terminal 3 installed in another facility, can provide support for another examiner who uses the internal examiner terminal 3 installed in the same facility as the ophthalmic examination apparatus 2.

For example, the examiner, who uses the internal examiner terminal 3 installed in the same facility as the ophthalmic examination apparatus 2, enters a message indicating a question, a doubt, or a problem using the operation device 312 and the GUI, etc. It is also possible for the examiner to enter a message with the imaging device or the audio input device 314. The entered message is sent to the external examiner terminal 5 (or the internal examiner terminal 3 installed in another facility) via the management apparatus 4. The controller 50 of the external examiner terminal 5 controls the display device 521 or the audio output device 523 to output the received message. With this, the examiner who uses the external examiner terminal 5 can perceive the message from the examiner who uses the internal examiner terminal 3.

The examiner who uses the external examiner terminal 5 can enter a reply message for the message from the examiner who uses the internal examiner terminal 3 by using the operation device 522 and the GUI, etc. It is also possible for the examiner to enter a message using the imaging device 51 or the audio input device 524. The entered message is sent to the internal examiner terminal 3 via the management apparatus 4. The controller 30 of the internal examiner terminal 3 controls the display device 311 or the audio output device 313 to output the received message. With this, the examiner who uses the internal examiner terminal 3 can perceive the reply message from the examiner who uses the external examiner terminal 5.

### <Second Example of Usage Mode>

The second example of the usage mode of the ophthalmic examination system 1 will be described. In the present example, it is possible to select an examiner having an attribute in accordance with the subject in order for the provision of assistance.

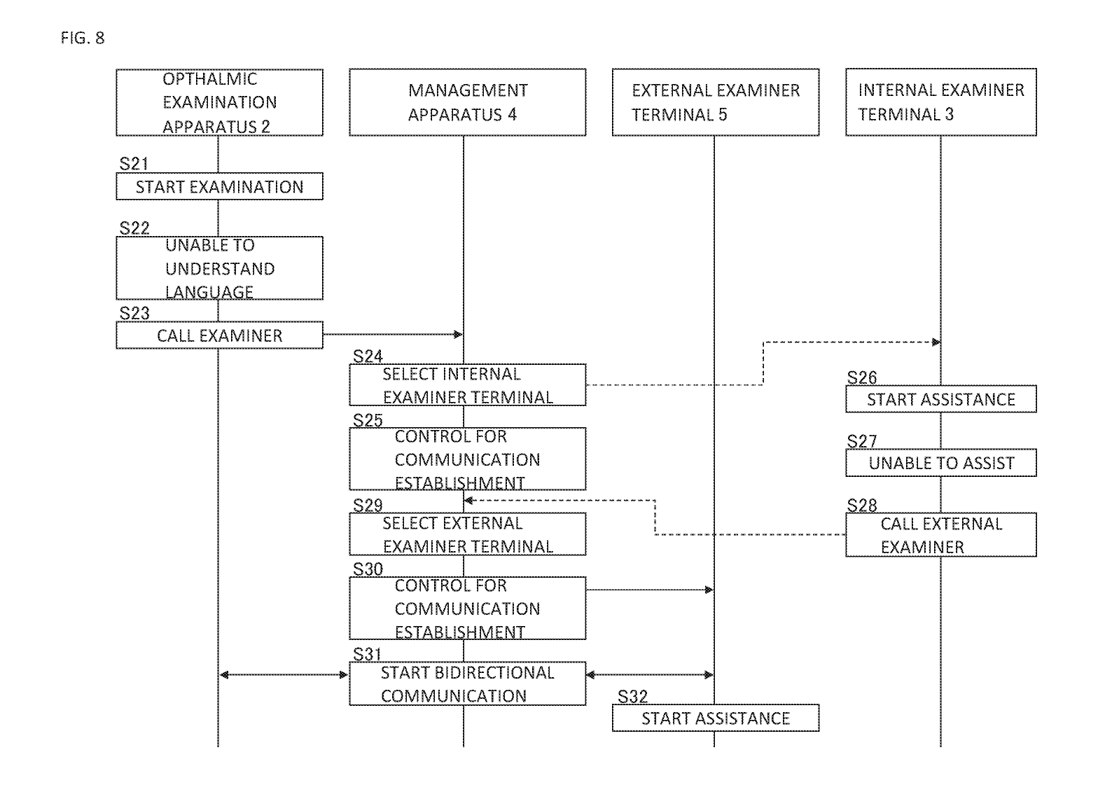

In FIG. 8, a specific example of the case in which an examiner is selected according to the language used by the subject will be described. It should be noted that the types of the attributes that can be referred to in the selection of examiner are not limited to language. For example, even when the examination cannot be performed smoothly due to a reason such as the fact that the subject being an elderly, the same processing can be performed.

Note that descriptions of steps and processes similar to those in the first example shown in FIG. 6 are omitted unless otherwise stated. In the example shown in FIG. 8, two examiner terminals, namely the internal examiner terminal 3 and the external examiner terminal 5, are involved; but examiner terminals that can be involved in the second example are not limited to this combination. In general, the second example can be realized with two or more examiner terminals arbitrarily selected from a plurality of examiner terminals including one or more internal examiner terminals 3 and one or more external examiner terminals 5.

### (S21: Start Examination)

The present example describes a case in which a subject, whose mother tongue is a language other than Japanese (e.g., English) and who has an insufficient understanding of Japanese, undergoes an examination using the ophthalmic examination apparatus 2 installed in a facility located in Japan. The examination of the subject's eye E using the ophthalmic examination apparatus 2 starts.

### (S22: Unable to Understand Language)

The ophthalmic examination apparatus 2 provides explanations and instructions (i.e., automatic navigation) of the examination for the subject in a language set in advance. In the present example, the ophthalmic examination apparatus 2 is set to provide explanations etc. in Japanese. The subject of the present example is unable to fully understand the automatic navigation given in Japanese.

### (S23: Call Examiner)

When the subject performs a predetermined operation or voice input to request examiner's assistance, the communication request generator 22b generates a communication request (i.e., call request) based thereon, and the communication device 24 sends the communication request and predetermined information to the management apparatus 4. Alternatively, the examination state information generator 22a of the ophthalmic examination apparatus 2 generates examination state information, and the communication request generator 22b generates a communication request (i.e., call request) based thereon. The communication device 24 then sends the call request and the predetermined information to the management apparatus 4.

### (S24: Select Internal Examiner Terminal)

The management apparatus 4 receives the examiner call request and the information transmitted from the ophthalmic examination apparatus 2 in step S23 by the communication device 42. At this stage, the selection device 412 selects any of the internal examiner terminal 3 used in the facility where the subject is undergoing the examination, as an examiner who will assist the subject.

### (S25: Control for Communication Establishment)

The communication controller 401 transmits a control signal, to the internal examiner terminal 3 selected in step S24, for establishing communication between this internal examiner terminal 3 and the ophthalmic examination apparatus 2.

### (S26: Start Assistance)

The internal examiner who uses the internal examiner terminal 3, whose communication with the ophthalmic examination apparatus 2 has been established by the control in step S25, checks the state of the subject. For example, the internal examiner checks the state by talking face to face with the subject. Alternatively, the internal examiner checks the state by communicating with the subject using the communication established in step S25.

### (S27: Unable to Assist)

If the internal examiner can cope with the state, the internal examiner continues providing the subject with the assistance. On the other hand, if the internal examiner determines that the state cannot be dealt with, the internal examiner proceeds to step S28 in order to ask an external examiner for assistance. The latter case will be described below.

### (S28: Call External Examiner)

The internal examiner performs a predetermined operation using the internal examiner terminal 3 in order to ask an external examiner for assistance. This operation may include, for example, an operation of selecting a language used by the subject. In the case where the call request or the information transmitted from the ophthalmic examination apparatus 2 to the management apparatus 4 in step S23 includes information indicating the language (e.g., English) used by the subject, the internal examiner need not perform the operation for selecting language. The communication device 33 transmits a communication request for calling an external examiner (and predetermined information) to the management apparatus 4.

### (S29: Select External Examiner Terminal)

The management apparatus 4 receives the communication request etc. transmitted from the internal examiner terminal 3 in step S28 by the communication device 42. At this stage, the selection device 412 selects an external examiner (or the external examiner terminal 5) who can use English by referring to the attribute information. At this time, the selection device 412 selects one external examiner according to the operating state of each external examiner who can use English.

### (S30: Control for Communication Establishment)

To the external examiner terminal 5 selected in step S29 (or, to the external examiner terminal 5 used by the external examiner selected in step S29), the communication controller 401 transmits a control signal for establishing communication between this external examiner terminal 5 and the ophthalmic examination apparatus 2.

### (S31: Start Bidirectional Communication)

By the control signal transmitted in step S30, bidirectional communication between the external examiner terminal 5 and the ophthalmic examination apparatus 2 is established. At this time, it is also possible to establish communication between the internal examiner terminal 3 and the external examiner terminal 5. Further, it is also possible to continue the established communication between the internal examiner terminal 3 and the ophthalmic examination apparatus 2.

### (S32: Start Assistance)

The external examiner, who is the user of the external examiner terminal 5 whose bidirectional communication with the ophthalmic examination apparatus 2 has started in step S31, gives remote assistance of the subject in English.

When there is a subject who cannot undergo examination smoothly, direct communication with this subject can be commenced with expedition according to the present example. This gives the subject a sense of security. Even when the problem cannot be solved by the direct communication, an external examiner suitable for the solution can be automatically selected to provide remote assistance. Therefore, it is possible to deal with various subjects without always having internal examiners of various attributes assigned to each facility.

### <Modification of Ophthalmic Examination Apparatus>

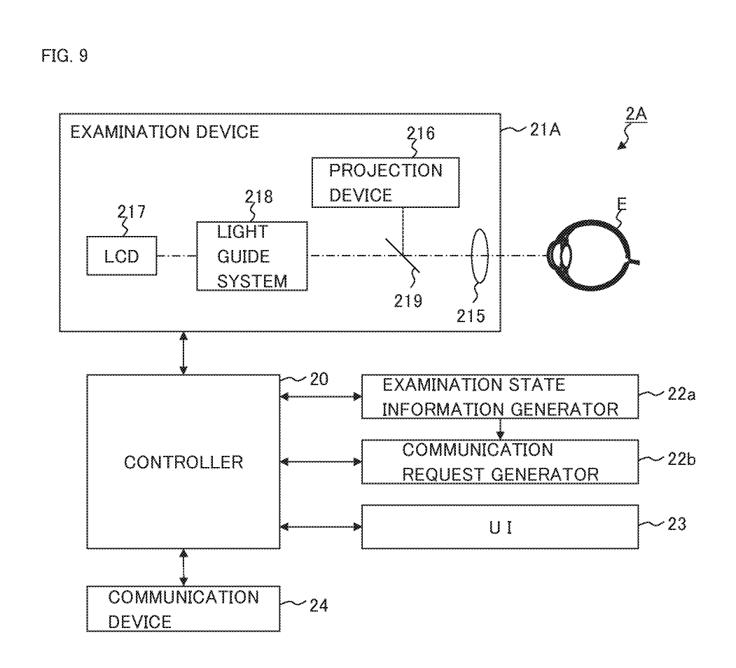

FIG. 9 shows an example in which the examination optical system and the information presenting optical system are separately arranged. In the examination device 21A of the ophthalmic examination apparatus 2A, the examination optical system used for the examination of the subject's eye E includes the optical element 215 applied to the subject's eye E and the projection system 216. The projection system 216 projects an examination light beam for examining the eye E onto the eye E via the optical element 215. The information presenting optical system for presenting information to the subject includes the LCD 217 (display device) and the light guide system 218. The light guide system 218 is configured to guide a display light beam output from the LCD 217 to the subject's eye E via the optical element 215. Examples of the information presented by the information presenting optical system include information automatically presented by the ophthalmic examination apparatus 2A, information transmitted from the internal examiner terminal 3 (e.g., an instruction from an internal examiner), and information transmitted from the external examiner terminal 5 (e.g., an instruction from an external examiner).

The optical path of the examination optical system and the optical path of the information presenting optical system are combined by the beam splitter 219. The beam splitter 219 may be, for example, a half mirror, a dichroic mirror, or a polarization beam splitter. Note that the elements other than the examination device 21 may be basically the same as those illustrated in FIG. 2.

Whether to configure the examination optical system and the information presenting optical system integrally as illustrated in FIG. 2 or configure them separately as illustrated in FIG. 9 is determined depending on, for example, the functions of the ophthalmic examination apparatus.

### <Presentation of Information by Examiner Terminal>

The configuration and the processing for presenting information to examiners will be described. As described above, an external examiner who is at the management center or at home can give assistance, from a remote location, to the subject undergoing examination using a predetermined ophthalmic examination apparatus 2. In addition, an internal examiner who is present in a facility different from the predetermined ophthalmic examination apparatus 2 can give assistance, from the remote location, to the subject undergoing examination using the ophthalmic examination apparatus 2. Furthermore, one examiner can support another examiner from a remote location.

Therefore, it is desirable to be able to present information to properly perceive the state of the subject or of another examiner at an appropriate timing with an appropriate interface. It is also possible to present information to two or more examiners in the same way. In this case, the management apparatus 4 (e.g., the transfer controller 402 therein) can perform control so as to synchronize the presentation of information for two or more examiner terminals.

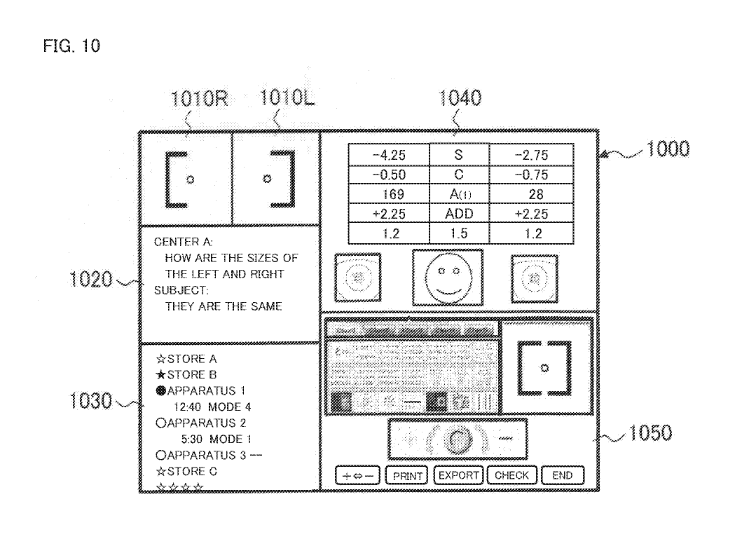

The controller (e.g., the controllers 50 or 30) of the examiner terminal (e.g., the external examiner terminal 5 or the internal examiner terminal 3) controls the display device (e.g. the display devices 521 or 311) to display the screen 1000 shown in FIG. 10. The screen 1000 includes the presentation information display areas 1010L and 1010R, the communication content display area 1020, the usage state display area 1030, the subject information display area 1040, and the presentation information operation area 1050.

In the presentation information display areas 1010L and 1010R, information presented to the left eye and information presented to right eye of the subject are displayed, respectively. In the present example, the ophthalmic examination apparatus 2 is configured to be able to present information to both left and right eyes of the subject at the same time. The ophthalmic examination apparatus 2 is provided with, for example, the examination optical system and the information presenting optical system for left eye and the examination optical system and the information presenting optical system for right eye independently of each other. For example, the ophthalmic examination apparatus 2 is provided with the optical head for left eye and the optical head for right eye. As another example, the ophthalmic examination apparatus 2 may be configured to split a light beam from a single examination optical system into two beams, and guide one of the two beams to the left eye and the other to the right eye. As still another example, the ophthalmic examination apparatus 2 may be configured to split a light beam from a single information presenting optical system into two beams, and guide one of the two beams to the left eye and the other to the right eye. The configuration capable of simultaneously presenting information to both left and right eyes is not limited to the above. The configuration capable of simultaneously presenting information to both left and right eyes may be arbitrary.

In the presentation information display areas 1010L and 1010R shown in FIG. 10, visual targets for the binocular visual function test are displayed. These visual targets are currently presented to the subject by the ophthalmic examination apparatus 2. Information indicating a visual target currently displayed (visual target information) is transmitted from the ophthalmic examination apparatus 2 to the management apparatus 4 and further transferred to the examiner terminal by the transfer controller 402. For example, this examiner terminal is the external examiner terminal 5. This examiner terminal may be the internal examiner terminal 3.

Based on the visual target information transferred from the management apparatus 4, the controller 50 of the external examiner terminal 5 specifies the type of the visual target currently presented by the ophthalmic examination apparatus 2, and displays the images representing the specified visual target on the presentation information display areas 1010L and 1010R. The images representing visual targets are stored in advance in a storage (not illustrated) in the controller 50 as a thumbnail, for example. Alternatively, the visual target information may include an image representing the visual target. When the visual target presented by the ophthalmic examination apparatus 2 is changed, the aforementioned transfer process is executed in real time. With this, the examiner who uses the external examiner terminal 5 can recognize the visual target currently presented by the ophthalmic examination apparatus 2 substantially in real time.

In the communication content display area 1020, the contents of communication between the examiner and the subject are displayed. More specifically, the contents of assistance provided from the examiner to the subject are displayed. Further, the contents of communication between the examiners can be displayed in the communication content display area 1020. The information displayed here may include at least one of the followings: information provided by the external examiner; information provided by the internal examiner; and information from the subject (e.g., the contents of questions, the contents of responses). In addition, information on the users (e.g., the external examiner, the internal examiner, the subject) may be switched and displayed in the communication content display area 1020. It is also possible to display information from one or more users in a time-dependent order.

In the example shown in FIG. 10, the contents of the question issued by the examiner in the management center “A” (e.g., the person who uses the screen 1000) and the contents of the subject's response to the question are displayed on the communication content display area 1020. It is also possible to convert the voice information issued by the examiner or the subject into text information by means of a known voice recognition technology. Further, text information that has been previously associated with a predetermined operation may be displayed.

In the usage state display area 1030, the names of a plurality of facilities, “shop A”, “shop B”, etc., in which the ophthalmic examination apparatuses 2 are installed are displayed. Furthermore, identification information, “apparatus 1”, “apparatus 2”, etc., of the plurality of ophthalmic examination apparatuses 2 installed in a facility (for example, the shop B) selected from among these facilities by the examiner or the management apparatus 4 is displayed.

In the example shown in FIG. 10, the symbol presented on the left side of the facility name indicates whether or not the facility is selected. For example, the white star symbols attached to the store A and the store C indicate that these facilities are not selected, and the black star symbol attached to the store B indicates that the facility is selected. Note that such a star symbol group can be configured as a radio button. If this is the case, the examiner can select a desired one from among these facilities.

Further, the symbol presented on the left side of the identification information of the apparatus installed in the selected shop B likewise indicates whether or not it has been selected by the examiner, the management apparatus 4, or the like. In the example shown in FIG. 10, the white circle symbol indicates that the corresponding apparatus is not selected, and the black circle symbol indicates that the corresponding apparatus is selected. In the example shown in FIG. 10, the ophthalmic examination apparatus 2 with the identification information “apparatus 1” installed in the shop B is selected as the ophthalmic examination apparatus 2 (or the subject) for which the examiner is in charge of assistance. Note that such a circle symbol group can be configured as a radio button. If this is the case, the examiner can select a desired one from among these ophthalmic examination apparatuses 2.

On the right side of the identification information of the ophthalmic examination apparatuses 2, time information (for example, the time when the assistance was started, the elapsed time from the commencement of the assistance, etc.) and the communication establishment state (e.g., communication mode) relating to the ophthalmic examination apparatus 2 are presented.

The communication mode will now be described. In the present embodiment, four communication modes can be selected and applied. Further, it is possible to shift from one communication mode to another communication mode in an arbitrary manner.

The communication mode 1 shows the state in which the subject is undergoing examination by himself or herself with the ophthalmic examination apparatus 2. In the communication mode 1, the subject is not receiving any assistance from either the internal examiners or the external examiners. As for the ophthalmic examination apparatus 2, no communication has been established with either the internal examiner terminals 3 or the external examiner terminals 5. As described above, even when the communication mode 1 is being applied, information (e.g., examination state information, information displayed on the screen 1000) from the ophthalmic examination apparatus 2 may be transferred to any of the examiner terminals. With this, the examination by the ophthalmic examination apparatus 2 can be monitored and the examiner can issue a communication request as necessary to start the support. Also, when the subject requests assistance, the examiner is capable of responding to the request with expedition.

The communication mode 2 shows the state in which the subject is undergoing examination while receiving assistance from an external examiner (or an internal examiner who is present at another facility). In the communication mode 2, communication between the ophthalmic examination apparatus 2 and the external examiner terminal 5 (or the internal examiner terminal 3 installed in another facility) is established. As described above, even when the communication mode 2 is being applied, information from the ophthalmic examination apparatus 2 can be sent to the internal examiner terminal 3 installed in the same facility as the ophthalmic examination apparatus 2. With this, the internal examiner who is present at the same facility can respond with expedition, for example, when it becomes necessary to directly assist the subject.

The communication mode 3 shows the state in which the subject is undergoing examination while receiving assistance from an internal examiner who is in the same facility as the ophthalmic examination apparatus 2. In the communication mode 3, communication between the ophthalmic examination apparatus 2 and the internal examiner terminal 3 installed in the same facility is established. As described above, even when the communication mode 3 is being applied, information from the ophthalmic examination apparatus 2 can be sent to the external examiner terminal 5 (or the internal examiner terminal 3 installed in another facility). With this, the external examiner (or an internal examiner who is present at another facility) can respond with expedition, for example, when a situation arises in which the internal examiner who is in the same facility as the ophthalmic examination apparatus 2 is unable to manage.

The communication mode 4 shows the state in which the subject is undergoing examination while receiving assistance from both an internal examiner who is in the same facility as the ophthalmic examination apparatus 2 and an external examiner (or an internal examiner who is present at another facility). In the communication mode 4, communication between three or more apparatuses including the ophthalmic examination apparatus 2 and two or more examiner terminals is established. In a typical example, communication is established between three apparatuses, namely the ophthalmic examination apparatus 2, the internal examiner terminal 3 installed in the same facility as the ophthalmic examination apparatus 2, and the external examiner terminal 5 (or the internal examiner terminal 3 installed in another facility).

The communication mode 4 can be applied, for example, when it is necessary for two or more examiners to discuss or when one examiner is giving another examiner a training. The transfer controller 402 of the management apparatus 4 is capable of executing control so as not to send the communication contents between the examiners to the ophthalmic examination apparatus 2. For example, the transfer controller 402 can perform the following control: control of transferring information from the internal examiner who is in the facility where the ophthalmic examination apparatus 2 is installed to the external examiner, only to the external examiner terminal 5; control of transferring information from the external examiner to the internal examiner, only to the internal examiner terminal 3; control of transferring information from the internal examiner to the subject, to the external examiner terminal 5 and the ophthalmic examination apparatus 2; control of transferring information from the external examiner to the subject, to the internal examiner terminal 3 and the ophthalmic examination apparatus 2; and control of transferring information output from the ophthalmic examination apparatus 2, to the external examiner terminal 5 and the internal examiner terminal 3.

Switching of communication modes can be executed, for example, by the management apparatus 4 that has received a communication request (e.g., call request, interruption request) from the subject or the examiner. In a specific example, when the subject issues a call request during the communication mode 1, the communication mode 1 gets switched to the communication mode 2, 3 or 4. The communication mode to be switched to can be determined, for example, according to the designation by the subject or the examiner, the contents of the examination state information, the aforementioned attributes, or the like.

When the internal examiner who is in the same facility issues an interruption request during the communication mode 1, the communication mode 1 is switched to the communication mode 3 or 4. In a similar manner, when the external examiner (or the internal examiner who is present at another facility) issues an interruption request in the communication mode 1, the communication mode 1 is switched to communication mode 2 or 4. Note that disconnection of the communication (that is, leaving the communication establishment state) can be carried out, for example, according to the selection of the examiner, the selection of the subject, the end of the examination, or the like.

In the subject information display area 1040, information relating to the subject is presented. In the example shown in FIG. 10, the measurement values (e.g., spherical powers, cylindrical powers, cylindrical axis angles, additional powers, visual acuity scores, etc.) of the both eyes of the subject, the anterior eye segment image of the both eye, the face image of the subject, and the like are displayed. In addition, it is also possible to present arbitrary kinds of information relating to the subject, such as name, age, sex, disease name, language used, level of difficulty in the performance of examination, and the like.

In the presentation information operation area 1050, a GUI (e.g., software keys) for operating information presented to the subject is provided. In the example shown in FIG. 10, the GUI for selecting a visual target to be presented to the subject (e.g., choices of visual targets, dials for changing powers or degrees, etc.) and various kinds of operation buttons (e.g., print button, export button) are provided. The examiner who uses the external examiner terminal 5 operates software keys or the like provided in the presentation information operation area 1050 by using the operation device 522. Similarly, the examiner who uses the internal examiner terminal 3 operates software keys or the like provided in the presentation information operation area 1050 by using the operation device 312.

### <Actions and Effects>

Actions and effects of the present embodiment will be described.

An ophthalmic examination system according to the present embodiment includes a plurality of ophthalmic examination apparatuses (2), a plurality of examiner terminals (internal examiner terminals 3 and/or external examiner terminals 5), a communication establishing device, an information transmission device, and an output device. Each of the plurality of ophthalmic examination apparatuses is used for examination (e.g., measurement, photography, etc.) of a subject's eye, and acquires data of the subject's eye. The plurality of examiner terminals is used by a plurality of examiners. The plurality of examiners includes, for example, an examiner who is in the same facility as the ophthalmic examination apparatus, an examiner who is in a facility different from the ophthalmic examination apparatus, an examiner in his or her home, and the like.

A communication establishing device is configured to be capable of establishing communication between at least three apparatuses. The three apparatuses include at least one of the plurality of ophthalmic examination apparatuses and at least two of the plurality of examiner terminals. In the above embodiment, the communication establishing device 41 functions as the communication establishing device. It should be noted that the communication establishing device need not be provided in the management apparatus. For example, the communication establishing device can be provided in any one or more of the plurality of ophthalmic examination apparatuses, any one or more of the plurality of examiner terminals, other ophthalmic apparatuses, other information processing apparatus, or the like.

The information transmitting device is configured to transmit at least part of information transmitted from one of the at least three apparatuses between which the communication has been established by the communication establishing device, to at least one of the other two or more apparatuses. In the above embodiment, the combination of the transfer controller 402 and the communication device 42 functions as the information transmitting device. Note that it is not necessary for the information transmitting device to be provided in the management apparatus 4. For example, it is possible to provide the information transmitting device in any one or more of the plurality of ophthalmic examination apparatuses, any one or more of the plurality of examiner terminals, other ophthalmic apparatuses, other information processing apparatus, or the like. In the case where the information transmitting device is provided in the ophthalmic examination apparatus, information can be directly transmitted from the ophthalmic examination apparatus to the examiner terminal or the like. Likewise, in the case where the information transmitting device is provided in the examiner terminal, information can be sent directly from the examiner terminal to the ophthalmic examination apparatus or another examiner terminal.

The output device is configured to output information transmitted by the information transmitting device. In the above embodiment, each of the LCD 214, the display device 231, and the audio output device 233 of the ophthalmic examination apparatus 2 functions as the output device. In addition, each of the display device 311 and the audio output device 313 of the internal examiner terminal 3 functions as the output device. Furthermore, each of the display device 521 and the audio output device 523 of the external examiner terminal 5 functions as the output device. It should be noted that the output device need not be a part of the ophthalmic examination apparatus or a part of the examiner terminal. For example, the output device may be a display device connected to the ophthalmic examination apparatus or a display device connected to the examiner terminal.

According to such an embodiment, it is possible to deal with various situations arising in remote examinations. For example, it is possible to provide prompt assistance for the subject according to the request of the subject or the judgment of the examiner. Further, it is possible to provide support for the examiner with expedition according to the request of the examiner or the judgment of another examiner. In addition, it is possible to issue an appropriate instruction at an appropriate timing according to the degree of understanding of the subject with regard to the examination contents. Further, in the case where the examiner is not accompanying the subject, it is possible to perceive the state of the subject and the progress state of the examination substantially in real time and to assist the subject. In addition, in the case where the examiner is accompanying the subject, it is possible to perceive the state of the same examiner and the progress state of the examination substantially in real time and to support the same examiner. It is also possible to assign an examiner having a necessary ability to appropriately provide assistance (e.g., an examiner with a high degree of expertise, an expert, an examiner who can use a foreign language, etc.) to the subject with felicity.

The ophthalmic examination system of the present embodiment may include a communication request receiving device configured to receive a communication request from any of the plurality of ophthalmic examination apparatuses or any of the plurality of examiner terminals. If this is the case, the communication establishing device may include a selection device. Based on the communication request received by the communication request receiving device, the selection device selects two or more apparatuses other than the apparatus which has transmitted the communication request, from among the plurality of ophthalmic examination apparatuses and the plurality of examiner terminals. In addition, the communication establishing device may be configured to establish communication between at least three apparatuses including the apparatus which has transmitted the communication request and the two or more apparatuses selected by the selection device. In the above embodiment, the communication devices 24, 33, 42, 54, etc. function as a communication request receiving device. Also, the selection device 412 functions as the selection device.

According to such a configuration, in response to a communication request (e.g., call request, interruption request, etc.) from any one of the ophthalmic examination apparatus or any one of the examiner terminals, it is possible to establish communication between the apparatus from which the request was originated and other apparatuses. For example, it is possible to select an internal examiner and/or an external examiner according to a request for assistance from the subject and to assign the selected examiner in charge of assisting the subject. In addition, an internal examiner and an external examiner can offer to provide support, on the basis of the state of the examination, the support situation by other examiners, or the like.

In the present embodiment, the communication establishing device may include a storage. In the storage, attribute information is stored in advance. The attribute information represents an attribute(s) of a user(s) of at least one of the plurality of examiner terminals. If this is the case, the communication request may include information relating to the attribute of the user. Furthermore, the selection device is configured to execute selection from the plurality of examiner terminals, based on the attribute information and the communication request received by the communication request receiving device. In the above embodiment, the storage 411 functions as the storage.

According to such a configuration, it is possible to select an examiner having the attributes conforming to the state of the subject, the state of the examiner, the state of examination, or the like, and then to assign the selected examiner to assist the subject or support the examiner. Note that if the examiner terminals and the examiners are always associated in a one-to-one-correspondence manner, it can be considered that the attributes of the examiner terminals and the attributes of the examiners are identical with each other. In the absence of such a one-to-one correspondence, for example, the examiner terminals and the examiners are associated with each other by referring to the examiner IDs input at the time of using the examiner terminals. Then, it is possible to make the selection of the examiner terminal (that is, the selection of the examiner) by referring to the association relationship on the basis of the input examiner IDs.

In the present embodiment, at least one of the plurality of ophthalmic examination apparatuses may include an examination state information generator, a communication request generator, and a communication request transmitting device. The examination state information generator is configured to generate examination state information representing the state of the examination of the subject's eye being performed using the ophthalmic examination apparatus. The communication request generator is configured to generate communication request based on the examination state information generated by the examination state information generator. The communication request transmitting device is configured to transmit the communication request generated by the communication request generator, to the communication request receiving device. In the above embodiment, the examination state information generator 22a functions as the examination state information generator, the communication request generator 22b functions as the communication request generator, and the communication device 24 functions as the communication request transmitting device.

According to such a configuration, it is possible to automatically generate and send a communication request according to the state of the subject, the state of the examination, or the like. As a result, it becomes possible to commence the assistance of the subject with expedition. In addition, the possibility of missing a subject requiring assistance is reduced.

In the present embodiment, each of the plurality of ophthalmic examination apparatuses and each of the plurality of examiner terminals may include a user interface. The user interface is configured to receive an instruction to transmit the communication request to the communication request receiving device. In the above embodiment, the user interface 23, 31, 52, etc. function as the user interface. Typically, the user interface includes the operation devices 232, 312 and 522, the audio input devices 234, 314 and 524, and the like. In addition, the interface may include the controllers 20, 30, 50, etc. that respectively control the user interface 23, 31, 52, etc.

With such a configuration, the subject and the examiner can send a communication request at a desired timing.

In the present embodiment, when the communication request receiving device receives a communication request from any of the plurality of ophthalmic examination apparatuses, the selection device may be configured to execute the following processes. First, the selection device determines whether or not communication of the first examiner terminal, which corresponds to the communication request out of the plurality of examiner terminals, has been established. In a case where it is determined that the communication of the first examiner terminal has not been established, the selection device selects the first examiner terminal. On the other hand, in a case where it is determined that the communication of the first examiner terminal has already been established, the selection device selects the second examiner terminal different from the first examiner terminal. Here, the first examiner terminal corresponding to the communication request may be any of the followings: an internal examiner terminal installed in the same facility as the ophthalmic examination apparatus that has issued the communication request; an internal examiner terminal (or an internal examiner) having a predetermined attribute; and an external examiner terminal (or an external examiner) having a predetermined attribute.

According to such a configuration, when an assist request is issued from the subject while the internal examiners in the same facility are supporting other subjects, the assist request can be sent to an internal examiner in another facility or an external examiner. Conversely, if a suitable external examiner and a suitable internal examiner in another facility are supporting other subjects or examiners, the assist request can be sent to the internal examiners in the same facility. With this, it is possible to promptly giving the assistance to the subject requiring assistance.

It is also conceivable that all internal examiners in the same facility, all suitable external examiners, and all suitable internal examiners in other facilities are engaged in correspondence with other subjects or examiners. If this is the case, for example, control can be executed to present a message to the subject indicating that it will take time until the assistance will be provided. In addition, control can be executed to present a message to the examiner indicating that the subject is waiting.

In the present embodiment, the information transmitting device may be configured to transmit at least part of the information communicated between the third examiner terminal among the plurality of examiner terminals and any of the plurality of ophthalmic examination apparatuses, to the fourth examiner terminal different from the third examiner terminal. If this is the case, the fourth examiner terminal may include the first display device and the first display controller. The first display controller is configured to display the information transmitted by the information transmitting device on the first display device.

In the above embodiment, when the third examiner terminal is the internal examiner terminal 3, the fourth examiner terminal is another internal examiner terminal 3 and/or the external examiner terminal 5. On the other hand, when the third examiner terminal is the external examiner terminal 5, the fourth examiner terminal is another external examiner terminal 5 and/or the internal examiner terminal 3. When the internal examiner terminal 3 is the fourth examiner terminal, the display device 311 functions as the first display device and the controller 30 functions as the first display controller. When the external examiner terminal 5 is the fourth examiner terminal, the display device 521 functions as the first display device and the controller 50 functions as the first display controller.

With such a configuration, the contents of information exchanged between a certain examiner and the subject can be provided to another examiner. As a result, additional assistance for the subject from another examiner and training of the examiner by another examiner can be realized in an appropriate manner.

In the present embodiment, the information transmitting device may be configured to transmit information representing a current communication establishment state by the communication establishing device, to the fifth examiner terminal among the plurality of examiner terminals. If this is the case, the fifth examiner terminal may include the second display device and the second display controller. The second display controller is configured to display the information transmitted by the information transmitting device on the second display device.

In the above embodiment, the fifth examiner terminal may be the internal examiner terminal 3 and/or the external examiner terminal 5. When the internal examiner terminal 3 is the fifth examiner terminal, the display device 311 functions as the second display device and the controller 30 functions as the second display controller. When the external examiner terminal 5 is the fifth examiner terminal, the display device 521 functions as the second display device and the controller 50 functions as the second display controller.

With such a configuration, the examiner can grasp what kind of assistance is being provided for the ophthalmic examination apparatus. As a result, additional assistance and training of the examiner can be implemented in an appropriate manner.

In the present embodiment, at least one of the plurality of ophthalmic examination apparatuses may include a visual target presenting device and a visual target information generator. The visual target presenting device is configured to present a visual target for visual acuity test to the subject's eye. The visual target information generator is configured to generate visual target information indicating the visual target presented by the visual target presenting device. Further, the information transmitting device may be configured to transmit the visual target information generated by the visual target information generator, to the sixth examiner terminal among the plurality of examiner terminals. In addition, the sixth examiner terminal may include the third display device and the third display controller. The third display controller is configured to display the visual target presented to the subject's eye on the third display device, based on the visual target information transmitted by the information transmitting device.

In the above embodiment, the examination devices 21, 21A of the ophthalmic examination apparatus 2 function as the visual target presentation device, and the examination state information generator 22a (or the controller 20, etc.) functions as the visual target information generator. Also, the sixth examiner terminal may be the internal examiner terminal 3 and/or the external examiner terminal 5. When the internal examiner terminal 3 is the sixth examiner terminal, the display device 311 functions as the third display device and the controller 30 functions as the third display controller. When the external examiner terminal 5 is the sixth examiner terminal, the display device 521 functions as the third display device and the controller 50 functions as the third display controller.

With such a configuration, the examiner can easily perceive the visual target currently presented to the subject's eye. As a result, assistance for the visual acuity test can be appropriately performed. In particular, it becomes possible to appropriately perform remote assistance by an examiner who is outside the facility where the subject's visual acuity test is being performed.

In the present embodiment, the third display controller may display a graphical user interface for selecting a visual target on the third display device. If this is the case, the sixth examiner terminal may include a visual target selection information generator. The visual target selection information generator is configured to generate visual target selection information indicating a visual target selected using the graphical user interface. The information transmitting device is configured to transmit the visual target selection information generated by the visual target selection information generator, to the ophthalmic examination apparatus which has generated the visual target information. The visual target presenting device of the same ophthalmic examination apparatus is configured to present, to the subject's eye, the visual target corresponding to the visual target selection information transmitted by the information transmitting device.

In the above embodiment, the presentation information operation area 1050 shown in FIG. 10 functions as the graphical user interface. In the presentation information operation area 1050, the GUI for selecting a visual target to be presented to the subject is provided. The GUI includes, for example, the choices of visual targets, the dials for changing powers or degrees, and the like. When the internal examiner terminal 3 is the sixth examiner terminal, the transmission information generator 32 (or the controller 30) functions as the visual target selection information generator. When the external examiner terminal 5 is the sixth examiner terminal, the transmission information generator 53 (or the controller 50) functions as the visual target selection information generator.

With such a configuration, it is possible to select and change visual targets to be presented to the subject's eye by operating the examiner terminal.

The contents of the assistance actually provided can be recorded for a predetermined purpose such as the training of an examiner (for example, an unskilled person) or the evaluation of an examiner. For example, the history of information exchanged between the examiner and the subject, the history of information exchanged between the examiners, and the history of the visual targets presented to the subject in the visual acuity test (e.g., sequential order of visual target presentation) can be recorded. Also, it is possible to record the examiner who is providing assistance (for example, recording as a moving image), or to record the subject who is receiving assistance.

The management apparatus 4 of the above embodiment can be utilized as an apparatus for managing a remote medical system including a plurality of ophthalmic examination apparatuses and a plurality of examiner terminals. Such a management apparatus may have the same configurations and/or the functions as those of the management apparatus 4 described in the above embodiment. For example, the management apparatus may include at least one of the communication establishing device and the information transmitting device.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ophthalmic examination system comprising:
a plurality of ophthalmic examination apparatuses;
a plurality of examiner terminals;
a communication establishing device capable of establishing communication between at least three apparatuses including at least one of the plurality of ophthalmic examination apparatuses and at least two of the plurality of examiner terminals;
an information transmitting device that transmits at least part of information transmitted from any of the at least three apparatuses between which the communication has been established by the communication establishing device to at least one of other two or more apparatuses; and
an output device that outputs information transmitted by the information transmitting device.

2. The ophthalmic examination system of claim 1, further comprising a communication request receiving device that receives a communication request from any of the plurality of ophthalmic examination apparatuses and the plurality of examiner terminals,
wherein the communication establishing device comprises a selection device that selects two or more apparatuses other than an apparatus which has transmitted the communication request out of the plurality of ophthalmic examination apparatuses and the plurality of examiner terminals, based on the communication request received by the communication request receiving device, and
the communication establishing device establishes communication between at least three apparatuses including the apparatus which has transmitted the communication request and the two or more apparatuses selected by the selection device.

3. The ophthalmic examination system of claim 2, wherein
the communication establishing device comprises a storage in which attribute information representing an attribute of a user of at least one of the plurality of examiner terminals is stored in advance,
the communication request comprises information on an attribute, and
the selection device executes selection from the plurality of examiner terminals based on the attribute information and the communication request received by the communication request receiving device.

4. The ophthalmic examination system of claim 2, wherein at least one of the plurality of ophthalmic examination apparatuses comprises:
an examination state information generator that generates examination state information representing state of an examination;
a communication request generator that generates the communication request based on the examination state information generated by the examination state information generator; and
a communication request transmitting device that transmits the communication request generated by the communication request generator to the communication request receiving device.

5. The ophthalmic examination system of claim 2, wherein each of the plurality of ophthalmic examination apparatuses and the plurality of examiner terminals comprises a user interface that receives an instruction to transmit the communication request to the communication request receiving device.

6. The ophthalmic examination system of claim 2, wherein when the communication request receiving device receives a communication request from any of the plurality of ophthalmic examination apparatuses, the selection device determines whether or not communication of a first examiner terminal corresponding to the communication request among the plurality of examiner terminals has been established, selects the first examiner terminal in a case where it is determined that the communication has not been established, and selects a second examiner terminal different from the first examiner terminal in a case where it is determined that the communication has been established.

7. The ophthalmic examination system of claim 1, wherein
the information transmitting device transmits at least part of information communicated between a third examiner terminal among the plurality of examiner terminals and any of the plurality of ophthalmic examination apparatuses to a fourth examiner terminal different from the third examiner terminal,
wherein the fourth examiner terminal comprises:
a first display device; and
a first display controller that displays the information transmitted by the information transmitting device on the first display device.

8. The ophthalmic examination system of claim 1, wherein
the information transmitting device transmits information representing a current communication establishment state by the communication establishing device to a fifth examiner terminal among the plurality of examiner terminals,
wherein the fifth examiner terminal comprises:
a second display device, and
a second display controller that displays the information transmitted by the information transmitting device on the second display device.

9. The ophthalmic examination system of claim 1, wherein
at least one of the plurality of ophthalmic examination apparatuses comprises:
a visual target presenting device that presents a visual target for visual acuity test to a subject's eye; and
a visual target information generator that generates visual target information indicating the visual target presented by the visual target presenting device, and
the information transmitting device transmits the visual target information generated by the visual target information generator to a sixth examiner terminal among the plurality of examiner terminals,
the sixth examiner terminal comprises:
a third display device, and
a third display controller that displays the visual target presented to the subject's eye on the third display device, based on the visual target information transmitted by the information transmitting device.

10. The ophthalmic examination system of claim 9, wherein
the third display controller displays a graphical user interface for selecting a visual target on the third display device,
the sixth examiner terminal comprises a visual target selection information generator that generates visual target selection information indicating a visual target selected using the graphical user interface,
the information transmitting device transmits the visual target selection information generated by the visual target selection information generator to an ophthalmic examination apparatus which has generated the visual target information, and
the visual target presenting device of this ophthalmic examination apparatus presents a visual target corresponding to the visual target selection information transmitted by the information transmitting device, to the subject's eye.

11. The ophthalmic examination system of claim 1, wherein
at least one of the plurality of ophthalmic examination apparatuses comprises:
an examination optical system comprising an optical element applied to a subject's eye and a projection system that projects an examination light beam for examining the subject's eye onto the subject's eye via the optical element;
an information presenting optical system comprising a display device and a light guide system that guides a display light beam output from the display device to the subject's eye via the optical element; and
a controller that displays information transmitted from any of the plurality of examiner terminals on the display device.
